(19) United States
(12) Patent Application Publication
(10) Pub. No.: US 2014/0220704 A1
(43) Pub. Date: Aug. 7, 2014

(54) ARTICLES COMPRISING TEMPLATED CROSSLINKED POLYMER FILMS FOR ELECTRONIC DETECTION OF NITROAROMATIC EXPLOSIVES

(71) Applicants: Howard Edan Katz, Owings Mills, MD (US); Hoyoul Kong, Timonium, MD (US); Sun Jia, Baltimore, MD (US); Jasmine Sinha, Baltimore, MD (US)

(72) Inventors: Howard Edan Katz, Owings Mills, MD (US); Hoyoul Kong, Timonium, MD (US); Sun Jia, Baltimore, MD (US); Jasmine Sinha, Baltimore, MD (US)

(73) Assignee: The Johns Hopkins University, Baltimore, MD (US)

(21) Appl. No.: 14/235,520

(22) PCT Filed: Aug. 3, 2012
(86) PCT No.: PCT/US2012/049460

§ 371 Date: Apr. 8, 2014

Related U.S. Application Data
(60) Provisional application No. 61/514,647, filed on Aug. 3, 2011.
(60) Provisional application No. 61/526,484, filed on Aug. 23, 2011.

(51) Int. Cl. G01N 33/22

(52) U.S. Cl. CPC G01N 33/227
USPC 436/501; 422/82.01; 427/240; 427/430.1; 427/420; 702/27

(57) ABSTRACT

The present invention provides devices and articles of manufacture comprising a low-voltage operable, highly sensitive, and selectively responsive polymer for the detection of nitroaromatic compounds, including explosives. Resistive devices were fabricated by simple spin-coating on flexible and transparent substrates as well as silicon substrates, and were stable under ambient temperature and oxygen levels before exposure to the nitroaromatics. After exposure to 2,4,6-trinitrotoluene (TNT), the devices showed increased conductance, even with pg quantities of TNT, accompanied by a confirming color change from colorless to deep red. The relative conductance increase per unit exposure is the highest yet reported for TNT. Aromatic anion salts, on the other hand, did not induce any electronic responses. Methods of making the articles and devices, and their use are also provided.

### REFERENCE TO RELATED APPLICATIONS

This application claims the benefit of U.S. Provisional Patent Application Nos. 61/514,647, filed on Aug. 3, 2011, and 61/526,484, filed Aug. 23, 2011, both of which are hereby incorporated by reference for all purposes as if fully set forth herein.

### BACKGROUND OF THE INVENTION

Sensors for trace detection of explosives have been investigated for environmental remediation and military and homeland defense applications because of terrorist threats. Among various nitroaromatic explosives, 2,4,6,-trinitrotoluene (TNT) is the most commonly used. In addition, TNT is a poisonous compound which can cause skin irritation and abnormal liver function.

Several techniques for detecting TNT have been reported, including mass spectrometry coupled with gas chromatography (GC-MS), ion mobility spectrometry (IMS), microcantilevers, Raman spectroscopy, X-ray imaging, surface acoustic wave devices, and polymer fluorescence changes. Some of these techniques are very expensive and have limited selectivity, sensitivity, and portability, or require exacting nanofabrication. There are also numerous examples of electrochemical sensing of TNT. These demonstrations generally involve highly nanostructured electrodes, rare biomaterials, and/or fluidic electrochemical cells.

Templating, also known in more specific cases as molecular imprinting, has become a widely used method for the preparation of polymeric materials which have the ability to bind a specific chemical species in pores left behind when the template is removed. In some cases, there have been claims of enhanced geometric specificity to the binding. Templated polymers are robust, inexpensive and easy to prepare, and have specificity suitable for use in various sensor applications.

However, there still exists a need for a low-voltage operable, highly sensitive devices which can act as an electronic sensor for nitroaromatic explosive molecules.

### SUMMARY OF THE INVENTION

In accordance with an embodiment, the present invention provides an article of manufacture comprising: a molecularly-imprinted polymeric material comprising a cross-linked, water-soluble polymer; at least one imidazole compound, and a binding site capable of selectively binding one or more nitroaromatic compounds; wherein when the material is contacted with one or more nitroaromatic compounds, increases the conductance of the material when in the presence of an electric current.

In accordance with another embodiment, the present invention provides a method of making the article of manufacture described above comprising: a) adding to a solvent a sufficient amount of an imidazole compound, the templating nitroaromatic compound, a cosolvent, a cross-linker, and a reversible addition fragmentation chain transfer (RAFT) reagent to make a mixture; b) adding to the mixture of a), a catalyst at a sufficient concentration; and c) polymerizing the mixture of b) by heating the mixture of b) to a temperature of between 70-80° C. for about 2 to 6 hours, preferably about 4 hours; and d) coating the article with the mixture of c).

In accordance with still a further embodiment, the present invention provides a chemoresistor capable of selectively binding one or more nitroaromatic compounds comprising: a) a silicon substrate; b) one or more gold electrodes; and c) the article of manufacture described above.

In accordance with a further embodiment, the present invention provides a computerized system identifying one or more nitroaromatic compounds of interest associated with one or more conductance profiles comprising: a) a server and a client connected by a network; b) an application connected to the server and/or the client by the network, the application configured for: c) receiving, by a computer, the conductance profile one or more nitroaromatic compounds of interest from the article of manufacture or chemoresistor described above; d) comparing by a computer, a library of known conductance profiles of one or more nitroaromatic compounds of interest to the conductance profile from the article of manufacture or chemoresistor described above; e) identifying, by the computer, the conductance profile from the article of manufacture or chemoresistor described above when it matches a known conductance profiles of one or more nitroaromatic compounds of interest in the library; and f) transmitting, by the computer, the identified nitroaromatic compound of interest in the chemoresistor to the client.

In accordance with yet another embodiment, the present invention provides a method for detecting the presence of a nitroaromatic compound of interest comprising: a) providing a sensor comprising an article of manufacture described above: a support substrate; a pair of electrodes comprising (i) a first electrode and (ii) a second electrode, wherein (i) and (ii) comprise the same or different materials, and wherein at least one of (i) and (ii) is at the substrate; wherein the polymeric material is disposed between the first electrode and the second electrode and is bonded to the first electrode and to the second electrode, thereby forming a nanojunction between the first and second electrodes; a detection means operably connected to the pair of electrodes, the detection means capable of detecting a change in the electrical resistance or in the capacitance of the sensor when the polymeric material binds with the nitroaromatic compound of interest; b) exposing the polymeric material of the sensor to an environment containing nitroaromatic compound of interest such that the nitroaromatic compound of interest contacts the polymeric material; c) allowing the polymeric material to bind with the nitroaromatic compound of interest, the binding of the nitroaromatic compound of interest resulting in the sensor undergoing a detectable change in electrical resistance or in capacitance; and d) detecting any change in the electrical resistance or in the capacitance of the sensor, the detected change being indicative of the presence of the nitroaromatic compound of interest in the environment.

### BRIEF DESCRIPTION OF THE DRAWINGS

FIG. 1 illustrates the reaction scheme and operational principle of the template polymer materials of the present invention.

FIG. 2 depicts UV-vis absorption spectra of the (a) polymer solution, (b) polymer solution exposed to TNT, and (c) polymer film before and after exposure to TNT.

FIG. 3 shows (a) Schematic representation for the humidity adjustable probe station setup. (b) Constant temperature and relative humidity (RH) changed between 0 and 100% by dry N₂ gas and wet air flow. (c) The instant current change of the polymer device from the RH change. (d) Enlarged graph of (c) to show near zero current level at 0% RH (e) RH and temperature change for (f) the current changes of the devices exposed to various polar and non-polar solvents after 1.0 minutes and (g) enlarged graph of (f).

FIG. 4 is a set of graphs which show (a) the current change of the polymer device caused by RH changes: (Upper) The RH and temperature and (lower) the current changes. (b-f) Current changes of the devices exposed to 2.5 μL of 1.0 mg TNT/mL IPA solution and pure IPA solvent after 1.2 minutes at near (b) 1.0, (c) 25.0, (d) 40.0, (e) 73.0, and (f) 100.0% RH conditions. Note different y-axes.

FIG. 5 is a pair of graphs, (a) conducting behavior of the polymer (synthesized by using CPDB RAFT reagent) device at 67.0% RH. (b) Current changes of the polymer (synthesized by using CPDB RAFT reagent) devices exposed to 6.0 μl of 1.0 mg TNT/ml IPA and pure IPA solvent after 1.2 minutes at 15.0% RH.

FIG. 6 shows graphs depicting (a) sensitivity of the polymer devices using TNT solutions of various concentrations near 0% RH: current changes of devices exposed to the various concentrations. (b) and (c) (enlarged graph of (b)) very low-voltage (−0.1 V) operable devices. (d) devices on the flexible or transparent substrates such as stainless steel, PET, and Kapton film. (e) 1.0 and (f) 0.01 mg nitroaromatics/ml IPA after 1.2 minutes.

FIG. 7 includes graphs depicting (a) the current change of the polymer devices exposed to 1.0 and 0.01 mg negatively charged non-nitroaromatic compounds (sodium benzoate and sodium benzenesulfonate)/mL IPA (near 1.5% RH). (b) (upper images) The optical microscopy images and (lower images) pictures of devices which were used in experiment (a).

FIG. 8 is a series of graphs showing current responses to 2.5 μL TNT solutions of various concentrations (a) 1.0, (b) 0.5, (c) 0.1, and (d) 0.05 mg/mL IPA and (e) IPA solvent dropped 4 times on the same probed area of each polymer device. (f) The constant 0% RH and temperature.

FIG. 9 is a series of photomicrographs depicting ((a), (b)) and ((d), (e)) SEM, and (c) and (f) optical microscopy images of the polymer devices exposed to the IPA (a, b, c) and TNT solutions (d, e, f).

FIG. 10 is a series of optical microscopy images of the polymer films exposed to the (a) IPA solvent and (b) TNT solution for 10 minutes.

FIG. 11 is a graph showing XRD data from the polymer films before and after exposure to the IPA solvent or TNT solution.

FIG. 12 is a schematic showing proposed reaction mechanisms of the polymers of the present invention with a TNT molecule.

FIG. 13 is a series of NMR spectrographs (¹H aromatic region) of (a) TNT (b) polymer solution of the present invention, (b) 0.01, (d) 0.1, (e) 0.5, and (f) 1.0 ml TNT solution added to the polymer solution of the present invention of (b), and (g) 1.0 ml polymer solution of present invention added to TNT solution of (a).

FIG. 14 shows (a) ¹H NMR titration spectra showing TNT (0-0.092 M) aryl proton (H_{T}) shift on increasing its concentration in polymer solution in CD₃OD. ((a)-inset) Proposed complex formation of TNT with imidazoles attached to the polymer. Note the assigned chemical shift changes on complexation, consistent with our NMR observations. (b) Expanded view of ¹H NMR titration spectra.

### DETAILED DESCRIPTION OF THE INVENTION

A description of preferred embodiments of the invention follows. It will be understood that the particular embodiments of the invention are shown by way of illustration and not as limitations of the invention. At the outset, the invention is described in its broadest overall aspects, with a more detailed description following. The features and other details of the compositions and methods of the invention will be further pointed out in the claims.

The interaction between TNT and imidazole groups bound to the crosslinked acrylate polymers of the present invention, was found for the first time to cause an increase in conductivity of the polymer, along with a color change to the polymer. Other nitroaromatics also caused related but distinct time-dependent conductivity changes in contrast to that observed for negatively charged non-nitroaromatic compounds. Morphological changes in the polymers of the present invention were observed as well. The TNT-imidazole binding phenomenon was observed for a concentrated solution of the polymer in CD₃OD using NMR spectroscopy, and an equilibrium binding constant on the order of 20-60 M⁻¹ was determined, showing the favorability of the association.

The present invention relates to an article of manufacture comprising the polymer film, such as, for example, a sensor comprising the polymer film as generally described above, the sensor is useful for detecting the presence of target nitroaromatic compounds, and to methods of making and methods of using the articles to detect the presence of such nitroaromatic compounds in the environment.

In accordance with an embodiment, the present invention provides a molecularly-imprinted polymeric material (MIP) comprising: a cross-linked, water-soluble polymer; at least one imidazole compound, and a binding site capable of selectively binding one or more nitroaromatic compounds; wherein when the material is contacted with one or more nitroaromatic compounds, increases the conductance of the material when in the presence of an electric current.

It will be understood by those of ordinary skill in the art, that a MIP is generally described as a plastic cast or mold of the molecule of interest, where recognition is based on shape, much like a lock and key. MIPs are made by adding the molecule of interest to a solution of binding molecules that can be chemically incorporated into a polymer. These binders usually have an affinity for the target and form a complex.

The interactions that hold these complexes together include Π-Π interactions, hydrogen bonding, metal-ligand binding, and even covalent bond formation, but they must be reversible. The binder must also have a chemical functionality that allows it to be irreversibly bound to polymers. Vinyl groups are a common functional group used to prepare many polymers, e.g., polyethylene, polystyrene, polyvinylalcohol, and polyvinylchloride. The target-binder complex is dissolved in excess matrix monomer (for example, styrene) and possibly other additives such as a cross-linker and porogens (solvents).

In a typical sensor fabrication, a nonflowing plastic mass, consisting of the matrix and binder, is obtained which is chemically bound to the polymer/cross-linker matrix and the target molecule. Removal of the target is possible since it is reversibly bound to the binder. The cavity it leaves behind is permanently has a shape compatible with the target.

As used herein, the term “water soluble polymer” can comprise any monomer suitable for use in the present invention. Suitable non-limiting examples of monomers that can be used for preparing a MIP of the present invention include methylmethacrylate, other alkyl methacrylates, alkylacrylates, ally or aryl acrylates and methacrylates, cyanoacrylate, styrene, α-methyl styrene, vinyl esters, including vinyl acetate, vinyl chloride, methyl vinyl ketone, vinylidene chloride, acrylamide, methacrylamide, acrylonitrile, methacrylonitrile, 2-acetamido acrylic acid; 2-(acetoxyacetoxy)ethyl methacrylate 1-acetoxy-1,3-butadiene; 2-acetoxy-3-butenenitrile; 4-acetoxystyrene; acrolein; acrolein diethyl acetal; acrolein dimethyl acetal; acrylamide; 2-acrylamidoglycolic acid; 2-acrylamido-2-methyl propane sulfonic acid; acrylic acid; acrylic anhydride; acrylonitrile; acryloyl chloride; (R)-α-acryloxy-β,β′-dimethyl-g-butyrolactone; N-acryloxy succinimide N-acryloxytri.s(hydroxymethyl)aminomethane; N-acryloly chloride; N-acryloyl pyrrolidinone; N-acryloyl-tris(hydroxymethyl)amino methane; 2-amino ethyl methacrylate; N-(3-aminopropyl)methacrylamide; (o, m, or p)-amino-styrene; t-amyl methacrylate; 2-(1-aziridinyl)ethyl methacrylate; 2,2′-azobis-(2-amidinopropane); 2,2′-azobisisobutyronitrile; 4,4′-azobis-(4-cyanovaleric acid); 1,1′-azobis-(cyclohexanecarbonitrile); 2,2′-azobis-(2,4-dimethylvaleronitrile); 4-benzyloxy-3-methoxystyrene; 2-bromoacrylic acid; 4-bromo-1-butene; 3-bromo-3,3-difluoropropane; 6-bromo-1-hexene; 3-bromo-2-methacrylonitrile; 2-(bromomethyl)acrylic acid; 8-bromo-1-octene; 5-bromo-1-pentene; cis-1-bromo-1-propene; β-bromostyrene; p-bromostyrene; bromotrifluoro ethylene; (±)-3-buten-2-ol; 1,3-butadiene; 1,3-butadiene-1,4-dicarboxylic acid 3-butenal diethyl acetal; 1-butene; 3-buten-2-ol; 3-butenyl chloroformate; 2-butylacrolein; N-t-butylacrylamide; butyl acrylate; butyl methacrylate; (o,m,p)-bromostyrene; t-butyl acrylate; (R)-carvone; (S)-carvone; (−)-carvyl acetate; cis 3-chloroacrylic acid; 2-chloroacrylonitrile; 2-chloroethyl vinyl ether; 2-chloromethyl-3-trimethylsilyl-1-propene; 3-chloro-1-butene; 3-chloro-2-chloromethyl-1-propene; 3-chloro-2-methyl propene; 2,2-bis(4-chlorophenyl)-1,1-dichloroethylene; 3-chloro-1-phenyl-1-propene; m-chlorostyrene; o-chlorostyrene; p-chlorostyrene; 1-cyanovinyl acetate; 1-cyclopropyl-1-(trimethylsiloxy)ethylene; 2,3-dichloro-1-propene; 2,6-dichlorostyrene; 1,3-dichloropropene; 2,4-diethyl-2,6-heptadienal; 1,9-decadiene; 1-decene; 1,2-dibromoethylene; 1,1-dichloro-2,2-difluoroethylene; 1,1-dichloropropene; 2,6-difluorostyrene; dihydrocarveol; (±)-dihydrocarvone; (−)-dihydrocarvyl acetate; 3,3-dimethylacrylaldehyde; N,N′-dimethylacrylamide; 3,3-dimethylacrylic acid; 3,3-dimethylacryloyl chloride; 2,3-dimethyl-1-butene; 3,3-dimethyl-1-butene; 2-dimethyl aminoethyl methacrylate; 2,4-dimethyl-2,6-heptadien-1-ol; 2,4-dimethyl-2,6-heptadienal; 2,5-dimethyl-1,5-hexadiene; 2,4-dimethyl-1,3-pentadiene; 2,2-dimethyl-4-pentenal; 2,4-dimethylstyrene; 2,5-dimethylstyrene; 3,4-dimethylstryene; divinyl benzene; 1,3-divinyltetramethyl disiloxane; 8,13-divinyl-3,7,12,17-tetramethyl-21H,23H-porphine; 8,13-divinyl-3,7,12,17-tetramethyl-21H,23H-propionic acid; 8,13-divinyl-3,7,12,17-tetramethyl-21H,23H-propionic acid disodium salt; 3,9-divinyl-2,4,8,10-tetraoraspiro[5,5]undecane; divinyl tin dichloride; 1-dodecene; 3,4-epoxy-1-butene; 2-ethyl acrolein; ethyl acrylate; 2-ethyl-1-butene; (±)-2-ethylhexyl acrylate; (±)-2-ethylhexyl methacrylate; 2-ethyl-2-(hydroxymethyl)-1,3-propanediol triacrylate; 2-ethyl-2-(hydroxymethyl)-1,3-propanediol trimethacrylate; ethyl methacrylate; ethyl vinyl ether; ethyl vinyl ketone; ethyl vinyl sulfone; (1-ethylvinyl)tributyl tin; m-fluorostyrene; o-fluorostyrene; p-fluorostyrene; glycol methacrylate (hydroxyethyl methacrylate); GA GMA; 1,6-heptadiene; 1,6-heptadienoic acid; 1,6-heptadien-4-ol; 1-heptene; 1-hexen-3-ol; 1-hexene; hexafluoropropene; 1,6-hexanediol diacrylate; 1-hexadecene; 1,5-hexadien-3,4-diol; 1,4-hexadiene; 1,5-hexadien-3-ol; 1,3,5-hexatriene; 5-hexen-1,2-diol; 5-hexen-1-ol; hydroxypropyl acrylate; 3-hydroxy-3,7,11-trimethyl-1,6,10-dodecatriene; isoamyl methacrylate; isobutyl methacrylate; isoprene; 2-isopropenylaniline; isopropenyl chloroformate; 4,4′-isopropylidene dimethacrylate; 3-isopropyl-a-a-dimethylbenzene isocyanate; isopulegol; itaconic acid; itaconalyl chloride; lead (II) acrylate; (±)-:linalool; linalyl acetate; p-mentha-1,8-diene; p-mentha-6,8-dien-2-ol; methyleneamino acetonitrile; methacrolein; [3-(methacryloylamino)-propyl]trimethylammonium chloride; methacrylamide; methacrylic acid; methacrylic anhydride; methacrylonitrile; methacryloyl chloride; 2-(methacryloyloxy)ethyl acetoacetate; (3-methacryloxypropyl)trimethoxy silane; 2-(methacryloxy)ethyl trimethyl ammonium methylsulfate; 2-methoxy propene (isopropenyl methyl ether); methyl-2-(bromomethyl)acrylate; 5-methyl-5-hexen-2-one; methyl methacrylate; N,N′-methylene bisacrylamide; 2-methylene glutaronitrite; 2-methylene-1,3-propanediol; 3-methyl-1,2-butadiene; 2-methyl-1-butene; 3-methyl-1-butene; 3-methyl-1-buten-1-ol; 2-methyl-1-buten-3-yne; 2-methyl-1,5-heptadiene; 2-methyl-1-heptene; 2-methyl-1-hexene; 3-methyl-1,3-pentadiene; 2-methyl-1,4-pentadiene; (±)-3-methyl-1-pentene; (±)-4-methyl-1-pentene; (±)-3-methyl-1-penten-3-ol; 2-methyl-1-pentene; alpha.-methyl styrene; t-a-methylstyrene; t-β-methylstyrene; 3-methylstyrene; methyl vinyl ether; methyl vinyl ketone; methyl-2-vinyloxirane; 4-methylstyrene; methyl vinyl sulfone; 4-methyl5-vinylthiazole; myrcene; t-β-nitrostyrene; 3-nitrostyrene; 1-nonadecene; 1,8-nonadiene; 1-octadecene; 1,7-octadiene; 7-octene-1,2-diol; 1-octene; 1-octen-3-ol; 1-pentadecene; 1-pentene; 1-penten-3-ol; t-2,4-pentenoic acid; 1,3-pentadiene; 1,4-pentadiene; 1,4-pentadien-3-ol; 4-penten-1-ol; 4-penten-2-ol; 4-phenyl-1-butene; phenyl vinyl sulfide; phenyl vinyl sulfonate; 2-propene-1-sulfonic acid sodium salt; phenyl vinyl sulfoxide; 1-phenyl-1-(trimethylsiloxy)ethylene; propene; safrole; styrene (vinyl benzene); 4-styrene sulfonic acid sodium salt; styrene sulfonyl chloride; 3-sulfopropyl acrylate potassium salt; 3-sulfopropyl methacrylate sodium salt; tetrachloroethylene; tetracyano ethylene; tetramethyldivinyl siloxane; trans 3-chloroacrylic acid; 2-trifluoromethyl propene; 2-(trifluoromethyl)propenoic acid; 2,4,4′-trimethyl-1-pentene; 3,5-bis(trifluoromethyl)styrene; 2,3-bis(trimethylsiloxy)-1,3-butadiene; 1-undecene; vinyl acetate; vinyl acetic acid; 4-vinyl anisole; 9-vinyl anthracene; vinyl behenate; vinyl benzoate; vinyl benzyl acetate; vinyl benzyl alcohol; 3-vinyl benzyl chloride; 3-(vinyl benzyl)-2-chloroethyl sulfone; 4-(vinyl benzyl)-2-chloroethyl sulfone; N-(p-vinyl benzyl)-N,N′-dimethyl amine; 4-vinyl biphenyl(4-phenyl styrene); vinyl bromide; 2-vinyl butane; vinyl butyl ether; 9-vinyl carbazole; vinyl carbinol; vinyl cetyl ether; vinyl chloroacetate; vinyl chloroformate; vinyl crotanoate; vinyl cyclohexane; 4-vinyl-1-cyclohexene; 4-vinylcyclohexene dioxide; vinyl cyclopentene; vinyl dimethylchlorosilane; vinyl dimethylethoxysilane; vinyl diphenylphosphine; vinyl 2-ethyl hexanoate; vinyl 2-ethylhexyl ether; vinyl ether ketone; vinyl ethylene; vinyl ethylene iron tricarbonyl; vinyl ferrocene; vinyl formate; vinyl hexadecyl ether; vinylidene fluoride; 1-vinyl imidizole; vinyl iodide; vinyl laurate; vinyl magnesium bromide; vinyl mesitylene; vinyl 2-methoxy ethyl ether; vinyl methyl dichlorosilane; vinyl methyl ether; vinyl methyl ketone; 2-vinyl naphthalene; 5-vinyl-2-norbornene; vinyl pelargonate; vinyl phenyl acetate; vinyl phosphonic acid, bis(2-chloroethyl)ester; vinyl propionate; 4-vinyl pyridine; 2-vinyl pyridine; 1-vinyl-2-pyrrolidinone; 2-vinyl quinoline; 1-vinyl silatrane; vinyl sulfone; vinyl sulfone (divinylsulfone); vinyl sulfonic acid sodium salt; o-vinyl toluene; p-vinyl toluene; vinyl triacetoxysilane; vinyl tributyl tin; vinyl trichloride; vinyl trichlorosilane; vinyl trichlorosilane (trichlorovinylsilane); vinyl triethoxysilane; vinyl triethylsilane; vinyl trifluoroacetate; vinyl trimethoxy silane; vinyl trimethyl nonylether; vinyl trimethyl silane; vinyl triphenyphosphonium bromide (triphenyl vinyl phosphonium bromide); vinyl tris-(2-methoxyethoxy)silane; vinyl 2-valerate and the like.

The term “cross-linking agent” as used herein, includes agents that impart rigidity or structural integrity to the polymers used in the articles and devices of the present invention are known to those skilled in the art, and include di-, tri- and tetrafunctional acrylates or methacrylates, divinylbenzene (DVB), alkylene glycol and polyalkylene glycol diacrylates and methacrylates, including ethylene glycol dimethacrylate (EGDMA, PEGDMA) and ethylene glycol diacrylate, vinyl or allyl acrylates or methacrylates, divinylbenzene, diallyldiglycol dicarbonate, diallyl maleate, diallyl fumarate, diallyl itaconate, vinyl esters such as divinyl oxalate, divinyl malonate, diallyl succinate, triallyl isocyanurate, the dimethacrylates or diacrylates of bis-phenol A or ethoxylated bis-phenol A, methylene or polymethylene bisacrylamide or bismethacrylamide, including hexamethylene bisacrylamide or hexamethylene bismethacrylamide, di(alkene) tertiary amines, trimethylol propane triacrylate, pentaerythritol tetraacrylate, divinyl ether, divinyl sulfone, diallyl phthalate, triallyl melamine, 2-isocyanatoethyl methacrylate, 2-isocyanatoethylacrylate, 3-isocyanatopropylacrylate, 1-methy:L-2-isocyanatoethyl methacrylate, 1,1-dimethyl-2-isocyanaotoethyl acrylate, tetraethylene glycol diacrylate, tetraethylene glycol dimethacrylate, triethylene glycol diacrylate, triethylene glycol dimethacrylate, hexanediol dimethacrylate, hexanediol diacrylate, and the like.

In accordance with one or more embodiments, free radical polymerization is preferred; however, monomers can also be selected that are polymerized cationically or anionically. Polymerization conditions should be selected that do not adversely affect the explosive chemical. Any UV or thermal free radical initiator known to those skilled in the art for free radical polymerization can be used to initiate this method. Examples of UV and thermal initiators include benzoyl peroxide, acetyl peroxide, lauryl peroxide, azobisisobutyronitrile (AIBN), t-butyl peracetate, cumyl peroxide, t-butyl peroxide, t-butyl hydroperoxide, bis(isopropyl)peroxy-dicarbonate, benzoin methyl ether, 2,2′-azobis(2,4-dimethylvaleronitrile), tertiarybutyl peroctoate, phthalic peroxide, diethoxyacetophenone, and tertiarybutyl peroxypivalate, diethoxyacetophenone, 1-hydroxycyclohexyl phenyl ketone, 2,2-dimethyoxy-2-phenyl-acetophenone, and phenothiazine, and diisopropylxanthogen disulfide.

The imidazole compounds used in the polymers of the articles of the present invention can be any imidazole containing compound suitable for use in the polymers of the present invention. Examples include N-vinylimidazole, alkyl-imidazoles, imidazole alcohols and esters and the like.

As used herein, the term “RAFT reagent” includes trimethylolpropane tris[3-dithiobenzoyloxypropionate] (TMPTDBP)) and 2-cyanoprop-2-yl-dithiobenzoate (CPDB), 4-cyano-4-[(dodecylsulfanylthiocarbonyl) sulfanyl]pentanoic acid, cyanomethyl methyl(phenyl) carbamodithioate, 4-cyano-4-(phenylcarbonothioylthio)pentanoic acid, 2-cyano-2-propyl dodecyl trithiocarbonate, 2-(dodecylthiocarbonothioylthio)-2-methylpropionic acid, cyanomethyl dodecyl trithiocarbonate, as well as other dithioesters, dithiocarbamates, and trithio-carbonates.

In accordance with another embodiment, the present invention provides a an article of manufacture comprising one or more molecularly-imprinted polymeric materials (MIPs) comprising: a cross-linked, water-soluble polymer; at least one imidazole compound, a RAFT reagent, and a binding site capable of selectively binding one or more nitroaromatic compounds; wherein when the material is contacted with one or more nitroaromatic compounds, increases the conductance of the material when in the presence of an electric current.

As used herein, the term “cosolvent” includes additional monomer solutions which add to the solubility of the monomers in polar solvents. Examples include trimethylammonium ethylmethacrylate chloride (TMAMA, 400 mg), other quaternary ammonium salt monomers such as trimethylammonium ethyl acrylate chloride or bromide, vinylbenzyltrimethylammonium chloride or bromide, and trimethylammonium ethyl acrylamide chloride or bromide.

In the case where the nitroaromatic compound of interest is an explosive compound, the template compound may be the explosive compound of interest itself, or it may be a non-explosive structural analog of the explosive compound. Examples of high-explosive compounds that may be targeted for detection include nitroaromatic explosive compounds such as trinitrotoluene (TNT), trinitrobenzene (TNB), or tetryl(2,4,6-trinitrophenyl-N-methylnitramine). Other examples of high-explosive compounds include nitrate explosives, such as urea nitrate or guanidine nitrate.

The term “structural analog,” as used herein, refers to a compound that shares molecular structural characteristics with a nitroaromatic compound of interest such that a molecularly-imprinted polymeric material that is imprinted with the structural analog will selectively bind with the nitroaromatic compound of interest. Non-explosive structural analogs of TNT have an acidic group (e.g., carboxylic acid) such that it can form a salt with the basic monomer(s). In this way, the monomers are held in place during polymerization. For example, non-explosive structural analogs of TNT include TMBA (2,4,6-trimethylbenzoic acid) and TCBA (2,4,6-trichlorobenzoic acid). Other structural analogs of TNT include benzoic acid derivatives having 1 to 3 substituents at the 2, 4, and/or 6 positions of the phenyl ring. These substituents should be similar in size to a nitro group and may be small aliphatic, halogen, or other electron withdrawing groups (e.g., methyl, ethyl, trifluoromethyl, etc.).

Non-explosive structural analogs of TNT also include nitroaromatics that contain only one or two nitro groups, such that it may avoid forming an irreversible Meisenheimer complex with the monomers. Examples of such structural analogs include nitrobenzene, ethylnitrobenzene, ethyldinitrobenzene, dinitrobenzene, nitrotoluene, dinitrotoluene, nitroxylene, dinitroxylene, 4-nitrophenol, and 2,4-dinitrophenol.

Polymerizations are generally conducted in bulk solution by the free-radical method. For bulk polymerization, typically about 0.10 to about 5.0 weight percent of the imidazole compound with a stoichiometric equivalent amount of the nitroaromatic compound, about 88 to about 93 weight percent monomer and about 1 to about 5 weight percent cross-linker, and about 1 weight percent of a free radical initiator such as azobia(isobutyronitrile) (AIBN) are dissolved in a polar organic solvent such as DMSO. The solution is heated to 70-80° C. for about 2-6 hours, preferably about 4. The reaction mixture is then dialyzed against water with a cutoff of about 3500 daltons.

To remove the nitroaromatic template compounds, the mixture is further dialyzed against a sodium bicarbonate buffer at a pH of between about pH 8 to pH 9, and aqueous ammonium solutions at a pH of about pH 10 to pH 12, preferably about pH 11.

In accordance with another embodiment, the present invention provides a solution comprising the polymeric material comprising: a cross-linked, water-soluble polymer; at least one imidazole compound, and a binding site capable of selectively binding one or more nitroaromatic compounds; wherein when the material is contacted with one or more nitroaromatic compounds, increases the conductance of the material when in the presence of an electric current.

The solutions comprising the polymeric material can be dissolved in organic solvents. In particular, the solutions comprising the polymeric material can be dissolved in polar organic solvents, including, for example, DMSO, methanol, ethanol, isopropanol, ethanol/acetonitrile/water, acetone/isopropanol, and the like.

The polymer solutions described herein, can be used to prepare thin films and coatings on a variety of surfaces and substrates to prepare articles of manufacture, such as devices and sensors of the present invention. Application of the solutions can be performed using any known means of applying polymer solutions. Examples of such applications include spin casting, drop casting and dip-coating.

The MIP polymers of the present invention can be prepared in a wide variety of forms ranging from powders to beads to macro structures such as plates, rods, membranes or coatings or other materials. A wide range of devices and sensors of the present invention can be produced from the MIP polymers described herein, and the type of sensor will depend on the conditions of use (e.g., spot monitoring, continuous monitoring, process monitoring, etc.).

The relative amounts of each of the above-described monomers used in the polymer solutions will vary depending upon the desired chemical or physical properties of the polymeric material. Increasing the relative amount of the basic monomers may yield polymeric materials with more rapid colorimetric reaction kinetics. However, providing too much of the basic monomers can result in loss of selectivity. As such, in some cases, the basic monomers may be provided in an amount in the range of 0.5-15 wt % (relative to the other reactants in the mixture), and in some cases, in the range of 1-10 wt %. The amount of water-soluble monomer in the mixture is sufficient to form water-soluble polymers. Balanced against the need for basic monomers that provide the colorimetric reaction, in some cases, a suitable ratio (by weight) of the water-soluble monomer to basic monomer may be in the range of 5:1 to 50:1; and in some cases, in the range of 10:1 to 30:1.

The cross-linking monomers are provided in an amount sufficient to cross-link the polymers to provide structural support and stability to the polymer. However, providing too much cross-linking monomers may result in polymers that are insoluble. As such, in some cases, the amount of cross-linking monomers in the reaction mixture is limited to 25 wt % (relative to the other reactants in the mixture) or less; and in some cases, 15 wt % or less; and in some cases, 10 wt % or less.

The polymerization reaction may be carried out in any conventional fashion (e.g., free radical polymerization initiated by UV irradiation or a free radical initiator such as azobisisobutyronitrile (AIBN)). In some cases, the polymerization may be a controlled free radical polymerization process to control the morphology, topology, and/or molecular weight distribution of the polymers (e.g., to a more narrow distribution). For example, the polymerization process may be carried out as a reversible addition fragmentation chain transfer (RAFT) process using a chain transfer agent (i.e., a RAFT reagent). Any of the various types of RAFT reagents known in the art, including dithioester agents, may be used in the RAFT process.

After the polymerization, the polymers may be further processed for purification, separation, isolation, and/or template removal. This processing may be performed in one or more steps, including filtration, centrifugation, washing, chromatographic separation, electrophoresis, and/or dialysis. Template removal may also be facilitated by a change in the pH or ionic strength of the solution.

In accordance with a further embodiment, the present invention provides an article of manufacture comprising the polymeric material comprising: a cross-linked, water-soluble polymer; at least one imidazole compound, and a binding site capable of selectively binding one or more nitroaromatic compounds; wherein when the material is contacted with one or more nitroaromatic compounds, increases the conductance of the material when in the presence of an electric current.

The articles of manufacture can be any substrate or article wherein the polymers of the present invention can be applied. Examples of articles include, sensors, electrodes, chemoresistors, dosimeters, and the like.

In accordance with still a further embodiment, the present invention provides a chemoresistor capable of selectively binding one or more nitroaromatic compounds comprising: a) a silicon substrate; b) one or more electrodes; and c) the polymeric material described above.

It will be understood by those of ordinary skill that the substrates and electrodes used in the chemoresistors and sensors of the present invention can be made using compositions and substances known in the art for such purposes. Substrates can include, for example, silicon and silicon dioxide, alumina, and ceramic substrates.

The electrodes used in the context of the present invention include conductive substances, including metals, such as gold, silver, molybdenum, palladium, niobium and platinum, for example.

In accordance with an embodiment, the present invention provides methods of making a sensor for detecting the presence of a target nitroaromatic compound comprising any electrode material, including, but not limited to examples of electrode materials described herein.

In accordance with an embodiment, the present invention provides methods for making a sensor for detecting explosives molecules, the methods comprising forming the pair of electrodes on the surface of the substrate by E-beam lithography. In another embodiment of a method for making a sensor according to the invention, a pair of electrodes is formed on the surface of the substrate by a break-junction technique comprising: depositing an electrode material as a wire on the surface of the substrate; and bending the substrate, thereby breaking the wire and forming a first and second electrode and an inter-electrode gap.

Yet another embodiment of the present invention comprises another method for making a molecular sensor for explosives comprising forming the pair of electrodes on the surface of the substrate by depositing a wire on the surface of the substrate; and applying an electrical current to the wire, thereby causing the wire metal to migrate and break, forming an inter-electrode gap. The wire can be made of any suitable metals, including, for example, gold.

In accordance with an embodiment, the present invention provides methods for detecting a target nitroaromatic compound, further comprising: generating an analog signal indicative of the change in the resistance of the resistors; converting the analog signal to a digital signal; transmitting the digital signal to a control module or data processing unit; analyzing the digital signal; and determining whether or not the digital signal exceeds a predetermined threshold value indicative of the presence of the nitroaromatic compound in the environment.

In accordance with yet another embodiment, the present invention provides a method for detecting the presence of a nitroaromatic compound of interest comprising: a) providing a sensor comprising: a support substrate; a pair of electrodes comprising (i) a first electrode and (ii) a second electrode, wherein (i) and (ii) comprise the same or different materials, and wherein at least one of (i) and (ii) is at the substrate; (iii) the polymeric material as described herein; wherein the polymeric material is disposed between the first electrode and the second electrode and is bonded to the first electrode and to the second electrode, thereby forming a connection between the first and second electrodes; a detection means operably connected to the pair of electrodes, the detection means capable of detecting a change in the electrical resistance or in the capacitance of the sensor when the polymeric material binds with the nitroaromatic compound of interest, wherein such means may also comprise one or more additional devices comprising the same or a related polymer material not exposed to the nitroaromatic under the test conditions; b) exposing the polymeric material of the sensor to an environment containing nitroaromatic compound of interest such that the nitroaromatic compound of interest contacts the polymeric material; c) allowing the polymeric material to bind with the nitroaromatic compound of interest, the binding of the nitroaromatic compound of interest resulting in the sensor undergoing a detectable change in electrical resistance or in capacitance; and d) detecting any change in the electrical resistance or in the capacitance of the sensor, the detected change being indicative of the presence of the nitroaromatic compound of interest in the environment.

In accordance with a further embodiment, the present invention provides a computerized system identifying one or more nitroaromatic compounds of interest associated with one or more conductance profiles comprising: a) a server and a client connected by a network; b) an application connected to the server and/or the client by the network, the application configured for: c) receiving, by a computer, the conductance profile one or more nitroaromatic compounds of interest from the article of manufacture or chemoresistor described above; d) comparing by a computer, a library of known conductance profiles of one or more nitroaromatic compounds of interest to the conductance profile from the article of manufacture or chemoresistor described above; e) identifying, by the computer, the conductance profile from the article of manufacture or chemoresistor described above when it matches a known conductance profiles of one or more nitroaromatic compounds of interest in the library; and f) transmitting, by the computer, the identified nitroaromatic compound of interest in the chemoresistor to the client.

It will be understood by those of ordinary skill in the art that the one or more conductance profiles identified can be used to generate a compound database. The database can be stored on computer readable media in a computer. The database can also be stored remotely over a network on a server and is accessible with at least one client computer attached to the network. The database is populated with information on one or more nitroaromatic compounds identified using the polymeric films or sensors described herein.

In accordance with an embodiment, the present invention provides at least one client computer which can be connected to at least one server computer over a network. At least one application can be connected to the at least one client computer and/or the at least one server computer over the network. The at least one application can comprise at least one target nitroaromatic compound determination module; and at least one nitroaromatic compound conductance profile database. It should be noted that the target nitroaromatic compound database can reside on the application, or outside the application. In addition, the application can reside on the client computer and/or the server computer. Furthermore, many additional databases and modules can be utilized by the application, and can reside on the application or outside the application.

### EXAMPLES

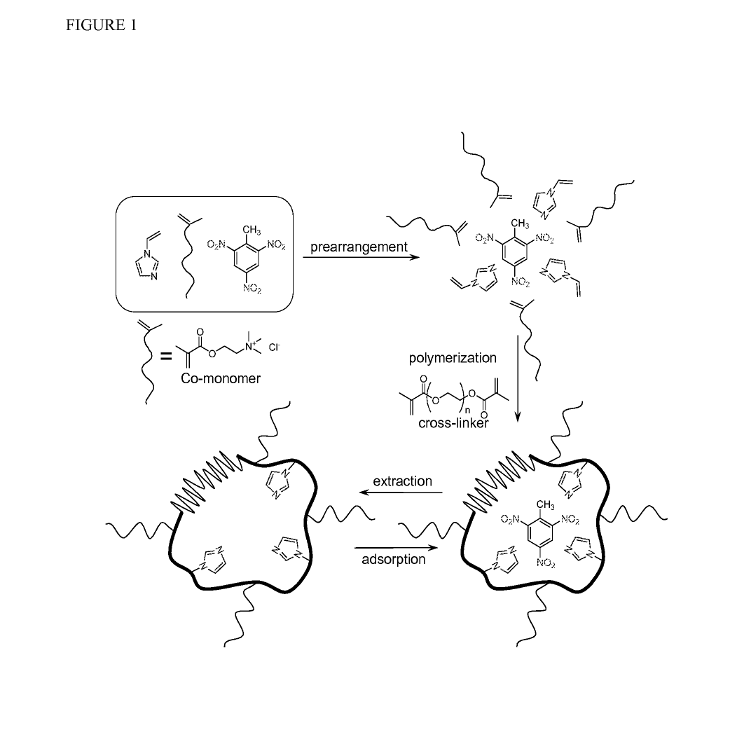

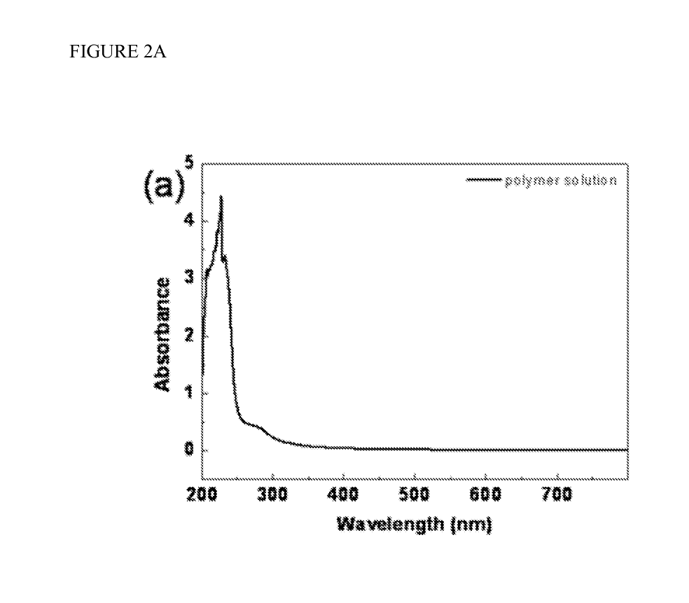

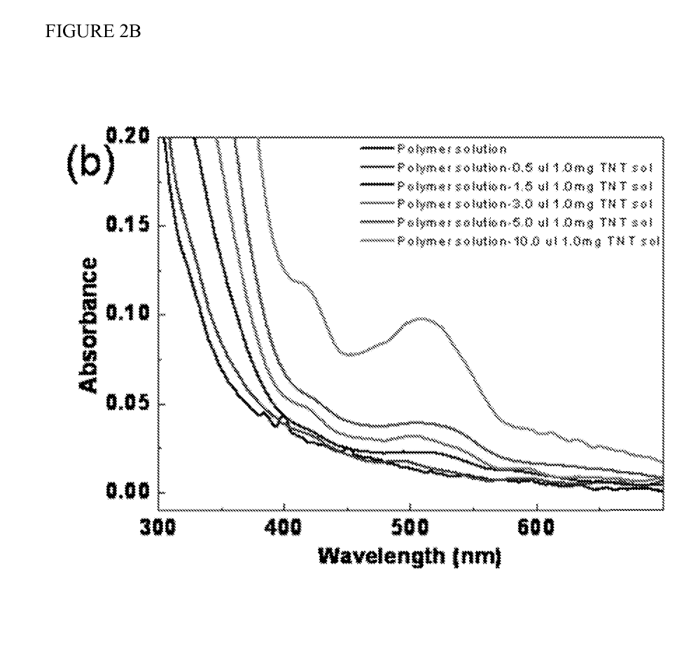

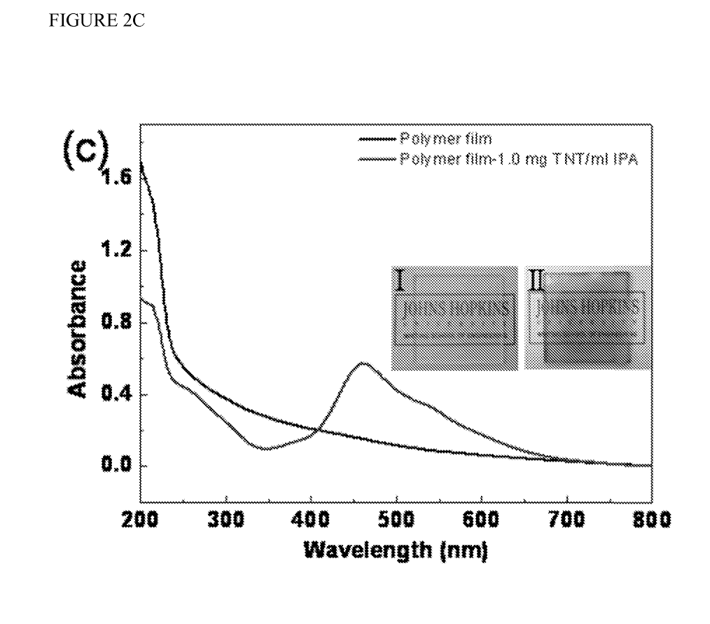

FIG. 1 shows a scheme depicting the synthesis, structure, and function of the polymers used in the articles of the present invention. The polymer synthesis was carried out using two different reversible addition fragmentation chain transfer (RAFT) reagents, firstly as reported in published patent application WO2010/078426 (trimethylolpropane tris[3-dithiobenzoyloxypropionate] (TMPTDBP)) and secondly using 2-cyanoprop-2-yl-dithiobenzoate (CPDB) as the RAFT reagent in order to verify the generality of the response towards TNT. Monomers undergo copolymerization in the presence of a cross-linker (polyethyleneglycol dimethacrylate (PEGDMA)) and TNT template. Subsequently, the TNT template and low molecular weight compounds are extracted by dialysis. Higher molecular weight, partially networked polymer remains, with free volume and functional groups compatible with nitroaromatics. The polymer was characterized by ¹H NMR (see below). The M_{w} for the CPDB polymer was about 3×10⁵ g/mol, and the TMPTDBP M_{w} was about an order of magnitude lower. The differential scanning calorimetry (DSC) thermograms for the polymers did not show any clear peaks indicative of phase transitions from room temperature to 300° C. (data not shown). UV-vis absorption spectra of the polymer (TMPTDBP) were recorded both in solution and in the film (FIG. 2). The UV-vis absorption maxima of the polymer is 227 nm in solution, and at even higher energy in film form, essentially transparent in the near UV-vis range. After exposure to some amount of TNT compound, the UV-vis absorption spectra of both polymer solution and film showed newly formed shoulder peaks at about 508 nm and 460 nm, respectively.

Polymer films were spin-coated on substrates including stainless steel, Kapton polyimide, and polyethylene terephthalate (PET), in addition to oxide-coated silicon (Si) substrates, and tested with 0.1 to 3 V applied between vapor-deposited interdigitated gold electrodes of total length 2.1 cm, spaced 0.25 mm apart. Polymer films were stable at least on the month time scale (data not shown). Data were taken with the TMPTDBP polymer, except for comparative data obtained on the CPDB polymer as noted.

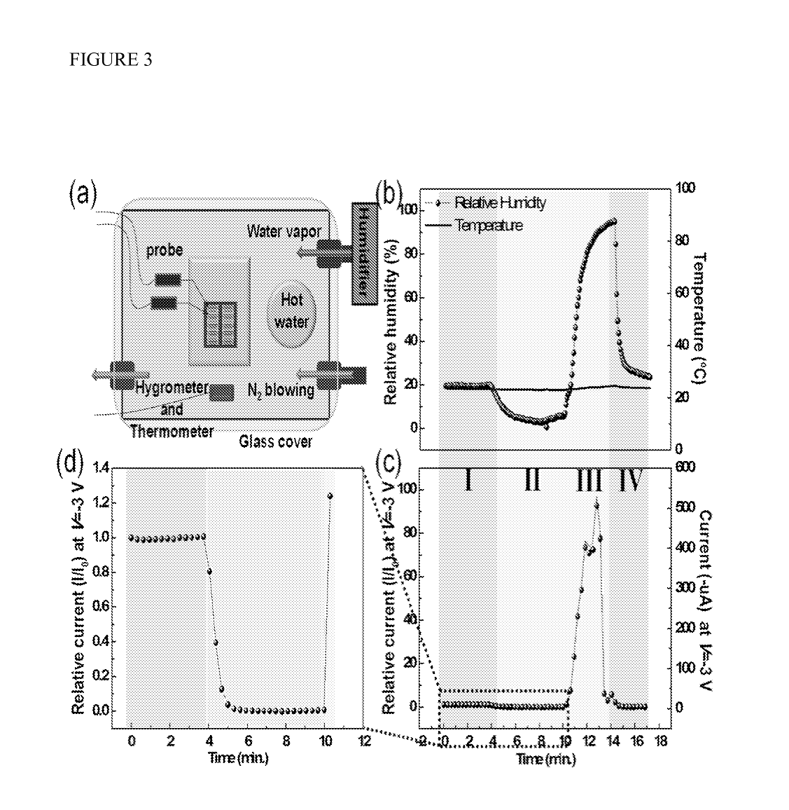

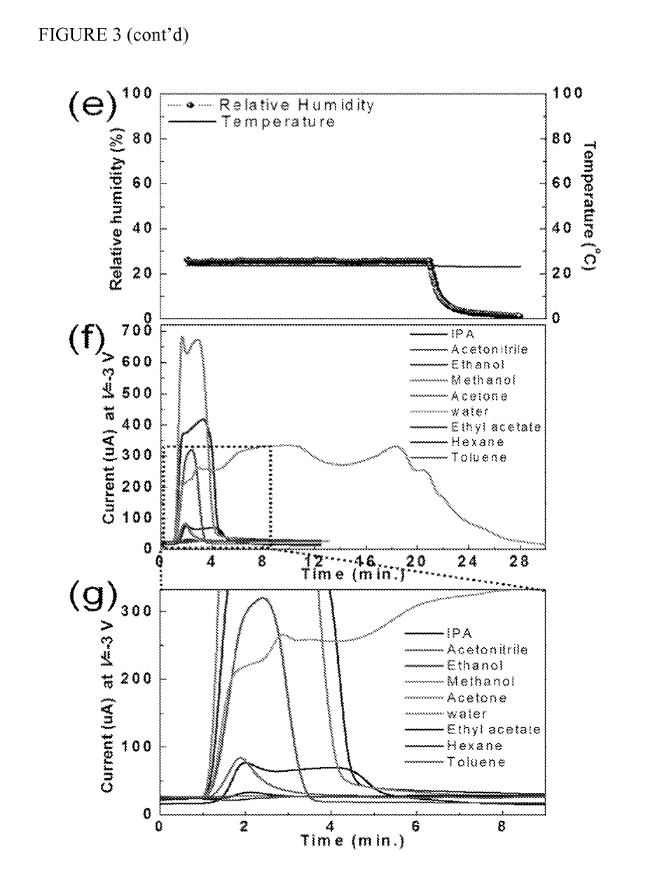

The probe station for I-V measurement was arranged in a relative humidity (RH)-adjustable and temperature-monitored chamber. The baseline current level was RH dependent as shown in FIG. 3, but stable at any given RH and recoverable on restoration of a prior RH. Sensitivity was greatest at the lowest RH, where the baseline current was lowest. It was also found that the hardness and morphology of the polymer film are RH-dependent.

When polar solvents were applied, currents increased: methanol (MeOH)>>ethanol (Et0H)>Acetonitrile≈water>>Acetone≈isopropyl alcohol (IPA)>>Ethyl acetate>toluene≈hexane (FIG. 3f). Again, original currents were recovered after solvent evaporation, and these current changes were readily distinguished from those induced by nitroaromatics. IPA was used as a solvent vehicle for nitroaromatics because of the modest induced current change by that solvent and the ready miscibility of TNT and the compatibility of the polyacrylate therewith. Responses were essentially instantaneous following nitroaromatic analyte application.

Synthetic procedures: Acrylate polymers were prepared according to a published procedure (WO2010/078426), which is summarized here. The preparation used N-vinylimidazole (25 mg, 5%) as a preformed salt with TNT, trimethylammonium ethylmethacrylate chloride (TMAMA, 400 mg) to increase solubility in polar solvents, and poly(ethylene glycol)dimethacrylate (PEGDMA) (MW 550, 25 mg, 5%) as a cross-linker in the presence of the RAFT reagents (TMPTDBP or CPDB) (20 mg). The polymerization reaction was carried in a sealed tube with dimethylsulfoxide (DMSO, 2 mL) as solvent and 2,2′-azobisisobutyronitrile (AIBN) (1%) as catalyst. The mixture was heated at 70-80° C. for 4 hours to promote polymerization. For purification of the polymer, the reaction mixture was dialyzed (MW cutoff of 3,500) against water. For template removal, the polymer solution was further dialyzed against a sodium bicarbonate buffer and aqueous ammonium solutions. ¹H NMR of the polymer using TMPTDBP (CD₃OD, δ ppm): 1.25, 1.67, 1.88, 2.29, 3.38, 3.44, 3.59, 3.66, 4.02, 4.12, 7.02, 7.27, 7.62, 7.80, 7.93.

¹H NMR of the polymer using CPDB (CD₃CN, δ ppm): 0.88, 1.27, 1.53, 1.62, 1.76, 3.02, 3.55, 3.68, 4.18, 4.23, 5.6, 6.02, 6.8, 7.14, 7.56, 7.62, 7.71, 7.94.

Measurement: ¹H NMR spectra were recorded on a Bruker AVANCE 400 MHz spectrometer (Billerica Mass.), with Me₄Si as an internal reference. The weight average molar mass (M_{W}) of the MIPs was determined using SLS (DAWN-HELEOS-II, Wyatt Technology, Goleta, Calif.) equipped with a laser operating at 658 nm. The measurements were carried out in water or acetonitrile at 22° C. The dn/dc were determined using Optilab T-rex (Wyatt Technology) at 658 nm. DSC measurement was performed using a TA Q20 instrument (TA Instruments, New Castle, Del.) under a nitrogen atmosphere at a heating rate of 5° C./min. The UV-vis spectra were recorded using a Cary 50 UV-vis spectrometer (Varian, Palo Alto, Calif.). The AFM measurements were performed in tapping mode with a Pico Plus scanning probe microscope. XRD measurements were carried out on a Phillips X-pert pro X-ray diffraction system (Phillips Systems, Netherlands). The polymer film thickness was measured by using SLOAN DEKTAK II (Veeco, Inc., Plainview, N.Y.).

Fabrication and characterization of chemoresistors of the present invention: Heavily n-type doped silicon wafers with 100 nm of thermally grown silicon dioxide layers were used as substrates. Wafers were cleaned by sonication in acetone and IPA. In addition, flexible or transparent substrates such as stainless steel, PET, and Kapton films were also used after air-blowing to remove dust. Gold electrodes were evaporated through a shadow mask. Polymer solution was spin coated at about 1500 rpm for 60 seconds on the substrates with gold electrodes. Films were annealed at 60° C. for removing methanol solvent. All the current-voltage (I-V) curves of devices were measured with an Agilent 4155C (Agilent Inc., Santa Clara, Calif.). The polymer devices were measured at −3 to +3 V, and the current at −3 V was extracted and plotted to show the current change (before and after exposure to analytes) versus time. For measuring, the temperature was maintained at constant values and the humidity was controlled by a humidifier and dry N₂ gas flow.

### Example 1

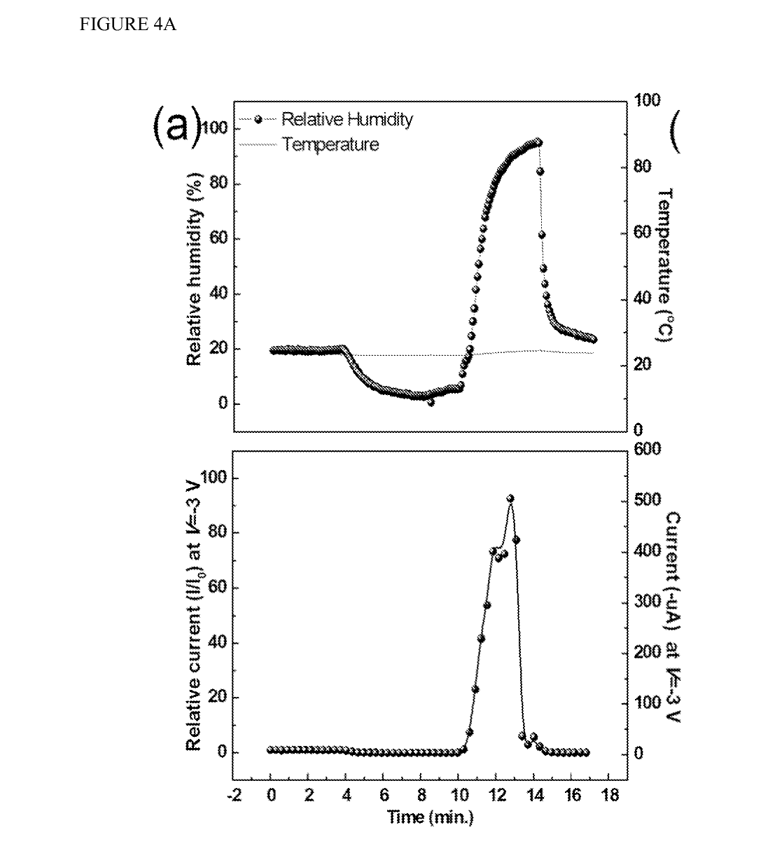

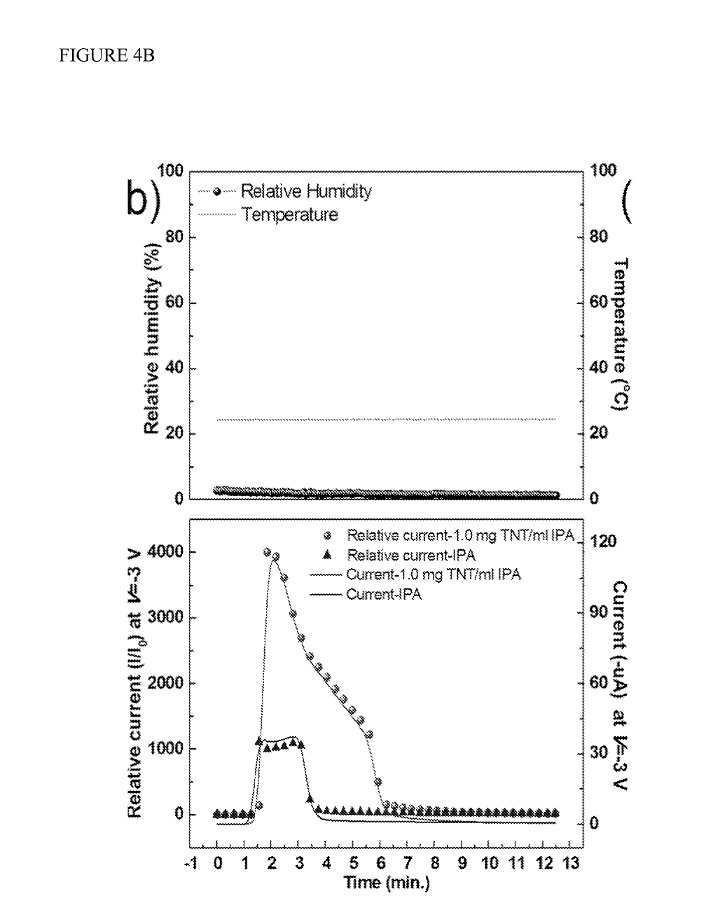

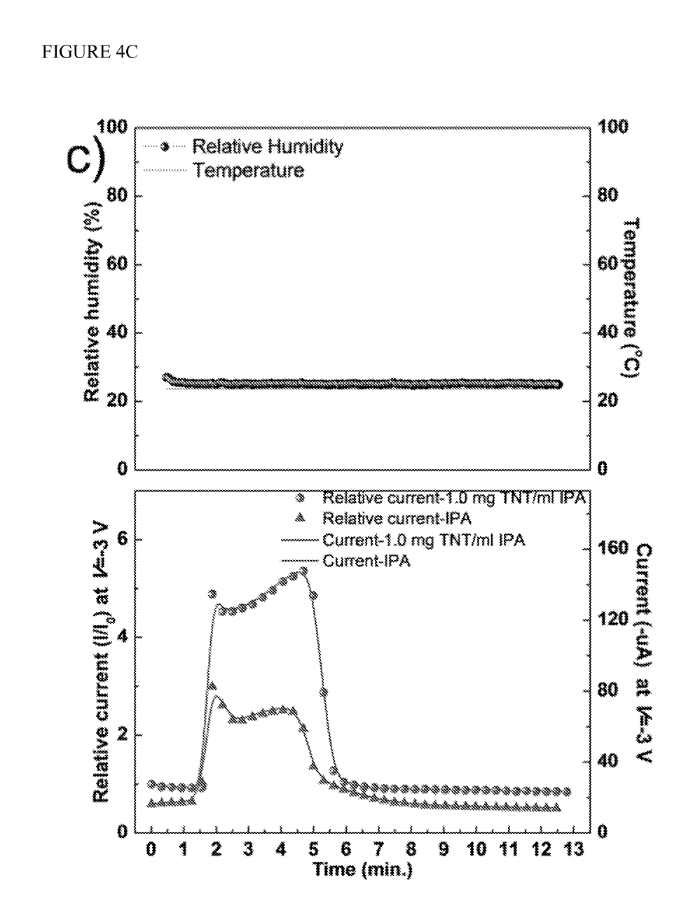

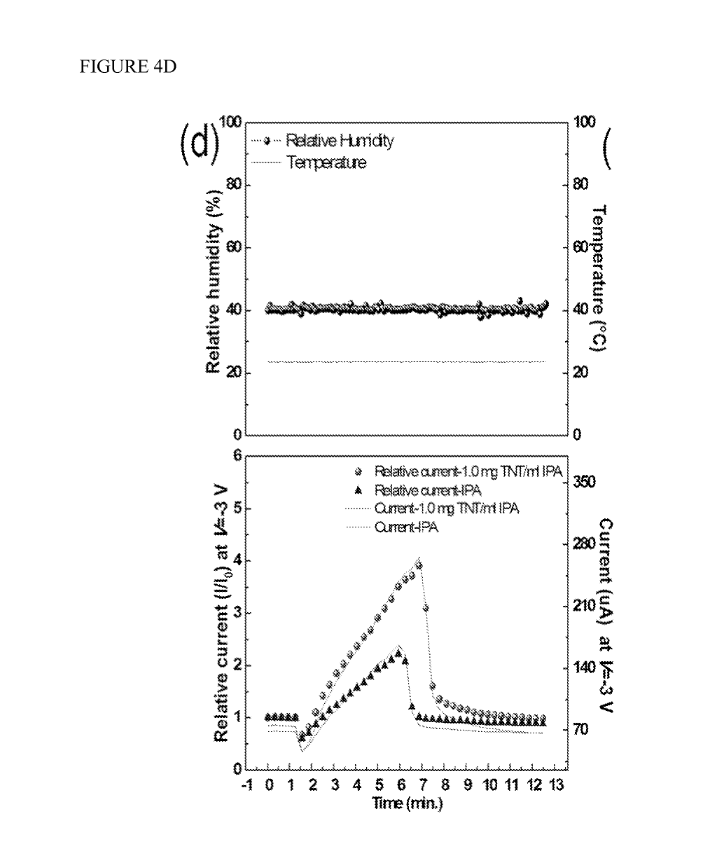

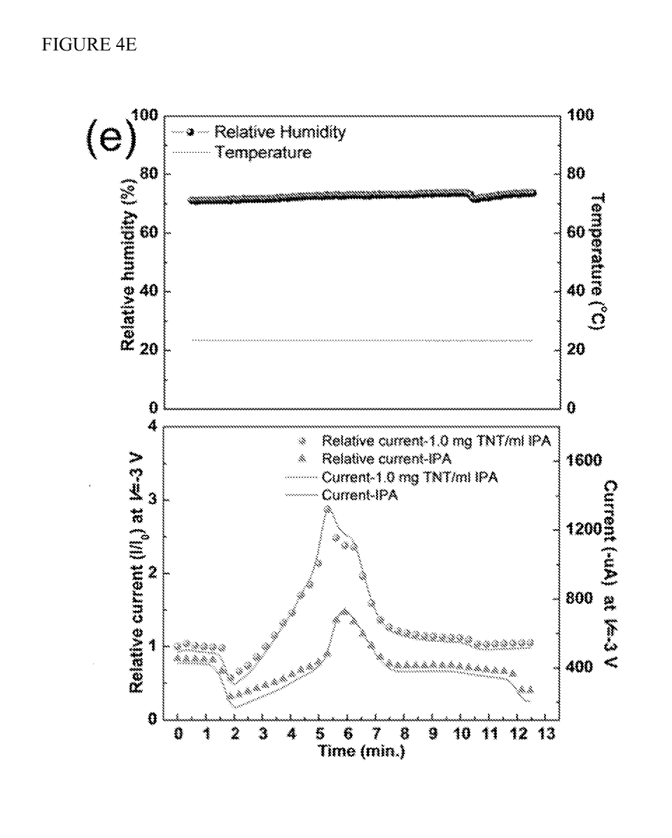

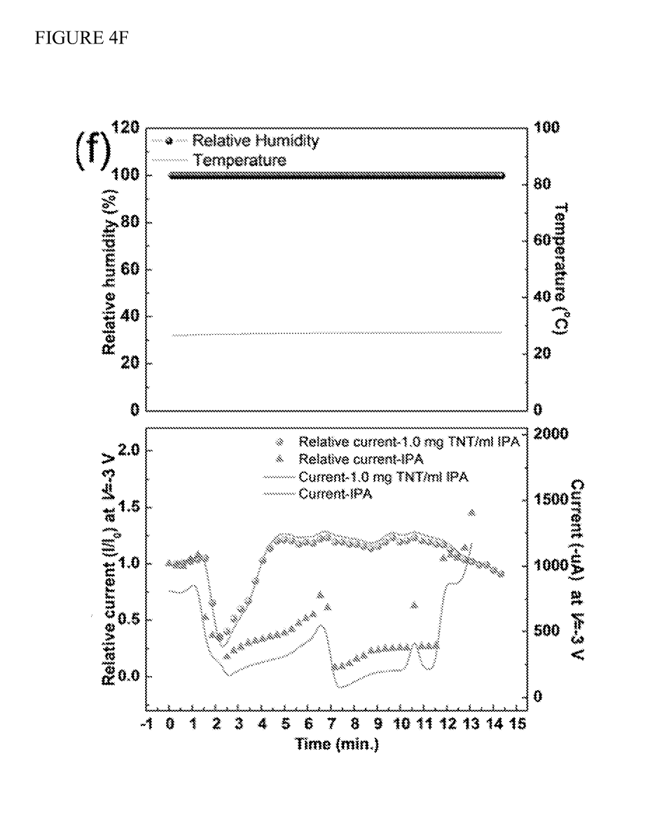

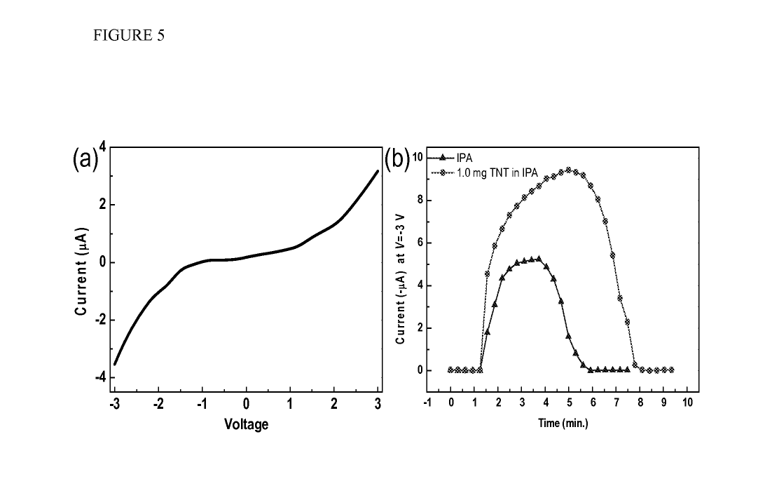

Exposures to nitroaromatics. The current changes of the polymers of the present invention (synthesized by using TMPTDBP RAFT reagent) devices after exposure to the TNT in IPA solution and to IPA solvent alone were investigated at various RH (FIG. 4). The measurements began after stabilization of the RH level. After 1.2 minutes, 2.5 μL of 1.0 mg TNT/mL IPA solution or IPA itself was dropped on the channel area of the polymer film. In all experiments, the devices exposed to the TNT solution showed much stronger response than those exposed to the pure solvent. Before and after exposure to the TNT, the devices of the present invention measured at higher RH showed much higher absolute current level. Although the absolute current of the devices measured at lower RH was lower, the relative current increase (current after exposure (I)/baseline current before exposure (I₀)) was much larger at lower RH than for those of the devices measured at higher RH, which means the TNT sensitivity is much higher. At very low RH near 1.0%, as soon as the TNT solution was dropped, the current was very rapidly increased from −0.032 μA to −115 μA (about 4000 times), a far greater change than that associated with the solvent. To the best of our knowledge, this is the highest conductance change yet reported in response to a given exposure to an explosive. At RH>40.4%, immediately when the TNT solution was dropped, the currents were briefly decreased, perhaps because of the lowered water activity, but between 40.0 and 73.0 RH, the TNT-induced current increase was then observed. In addition, the device of the polymer synthesized by using CPDB RAFT reagent also showed similar and reproducible current response to the TNT analyte solution (FIG. 5).

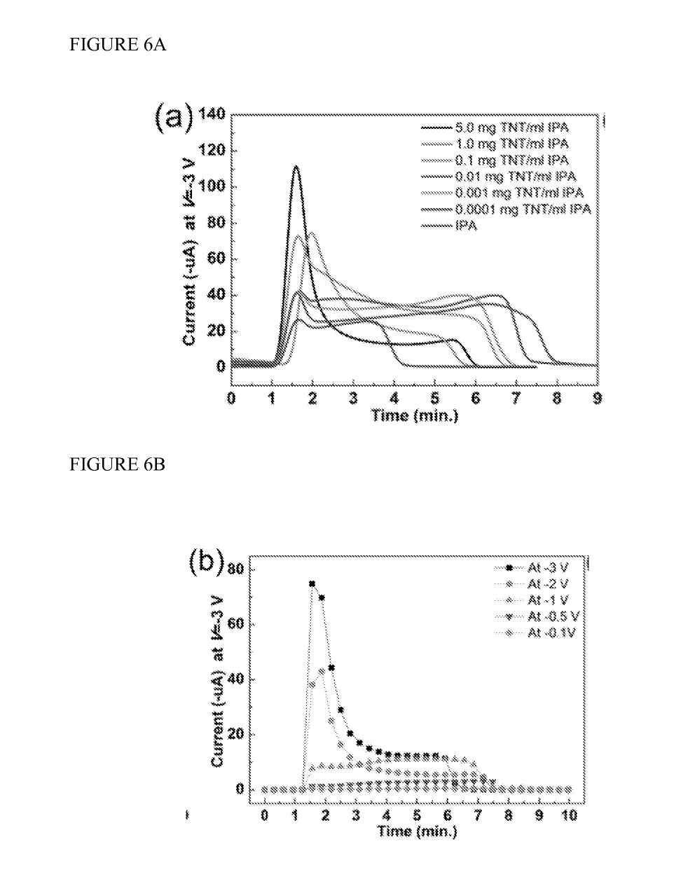

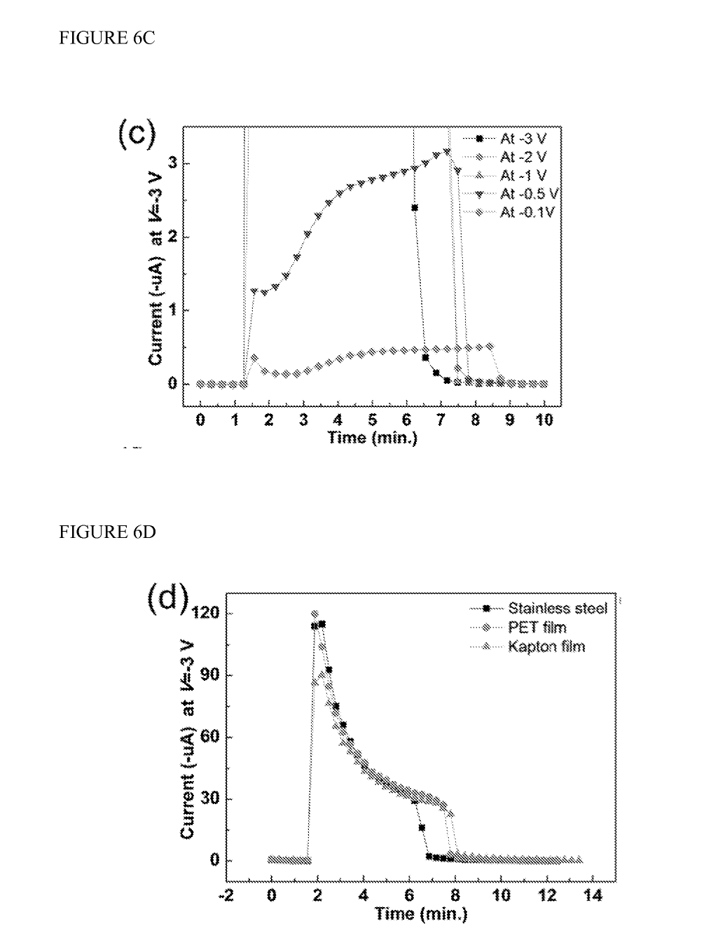

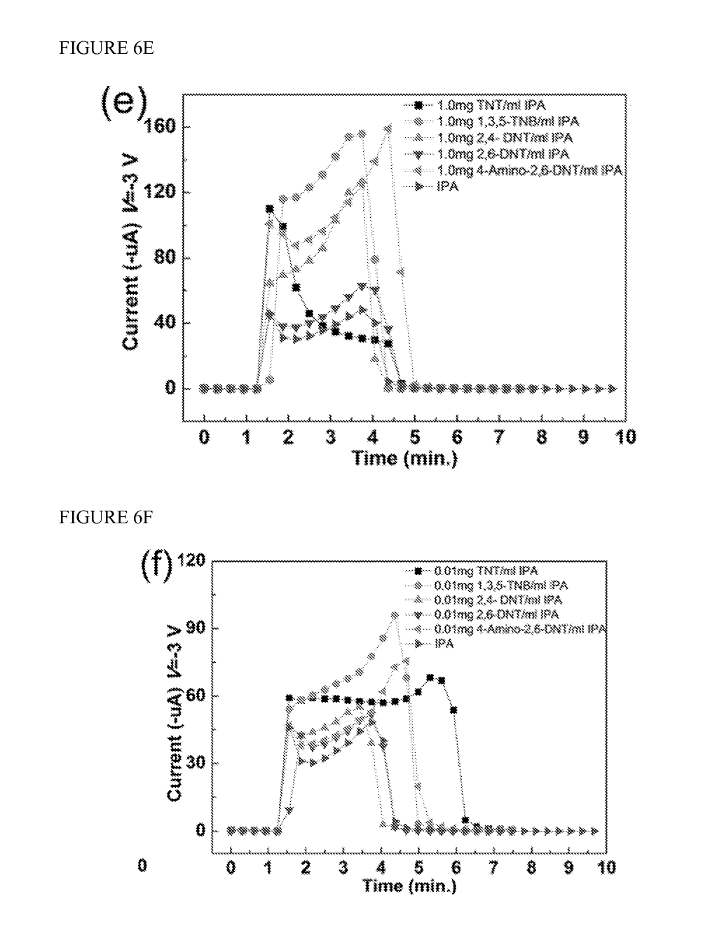

The sensitivity of the TMPTDBP polymer devices was investigated using various concentrations of the TNT solutions (FIG. 6a). Consistent current changes were observed. At higher TNT concentrations, the initial current change was much larger. In contrast, at lower TNT concentrations, although the initial current signal was much lower, the current was eventually increased over a longer time.

Devices exposed to TNT were investigated at various voltages from −3.0 to −0.1 V versus a grounded electrode (FIGS. 6b and 6c). Detection was accomplished at all voltages in the range, and at lower voltage, the time of elevated current was longer.

The films on flexible or transparent substrates such as stainless steel, PET film, and Kapton film (FIGS. 6d) was tested. The substrates were used without any surface treatments or special cleaning except for air blowing for removing dust. All devices showed current changes similar to what was observed on Si substrates. The PET film with polymer layer was flexible and transparent. After exposure to the TNT analyte, the transparent PET film became obviously red.

To investigate how long the polymer devices can be stored before attempting TNT detection, multiple devices were fabricated at the same time, and measured each device exposed to the TNT analyte after different storage times at 17% RH. The current change after exposure to the TNT analyte was reproducible (data not shown).

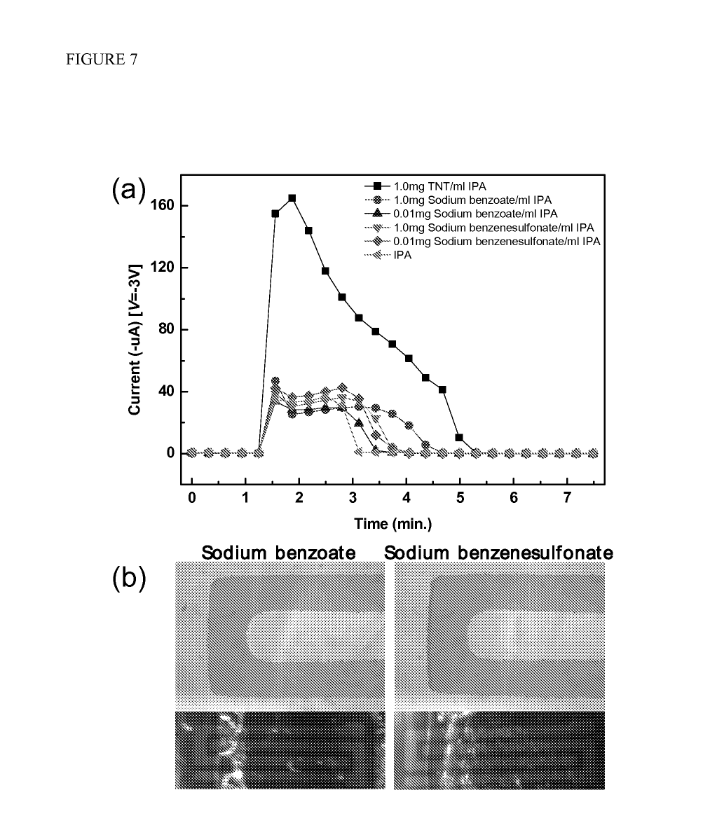

The reactivity of the devices to a series of nitroaromatics including TNT, 1,3,5-TNB, 2,4-DNT, 2,6-DNT, and 4-amino-2,6-DNT, were investigated (FIGS. 6e-6f). The apparent morphological change of the polymer film was observed by optical microscopy only with TNT exposure. In addition, the electrical changes were investigated using solution concentration levels of 1.0 and 0.01 mg nitroaromatics/mL IPA. TNT gave one of the strongest initial responses, with other nitroaromatics showing similar or less initial response. Devices exposed to TNT showed more sharply dropped current after the peaks at the higher concentration. Thus, the polymers of the present invention detect nitroaromatics in general, with TNT giving a distinct current evolution at the higher concentration. The polymer devices exposed to negatively charged aromatic salts such as sodium benzoate and sodium benzenesulfonate were also investigated. Although these aromatic anions have similar molecular shape and size compared to nitroaromatic explosives, there was no distinct current response, and no morphological or color changes when the polymer devices were exposed to the non-nitroaromatic compounds, in contrast to that observed for nitroaromatic explosives (FIG. 7).

To investigate the effect of the polymer thickness on the TNT sensitivity, many devices with a variety of thicknesses (0.9 to 24.1 μm) were prepared with different casting methods (spin or drop) using different concentration of the polymer solution (5 to 30%). The device with thinner polymer film showed larger relative current increase after exposure to TNT analyte. The polymer device with 0.9 μm thickness film exposed to 2.5 μL of 1.0 mg TNT/mL IPA showed 83,000 times increased current (data not shown).

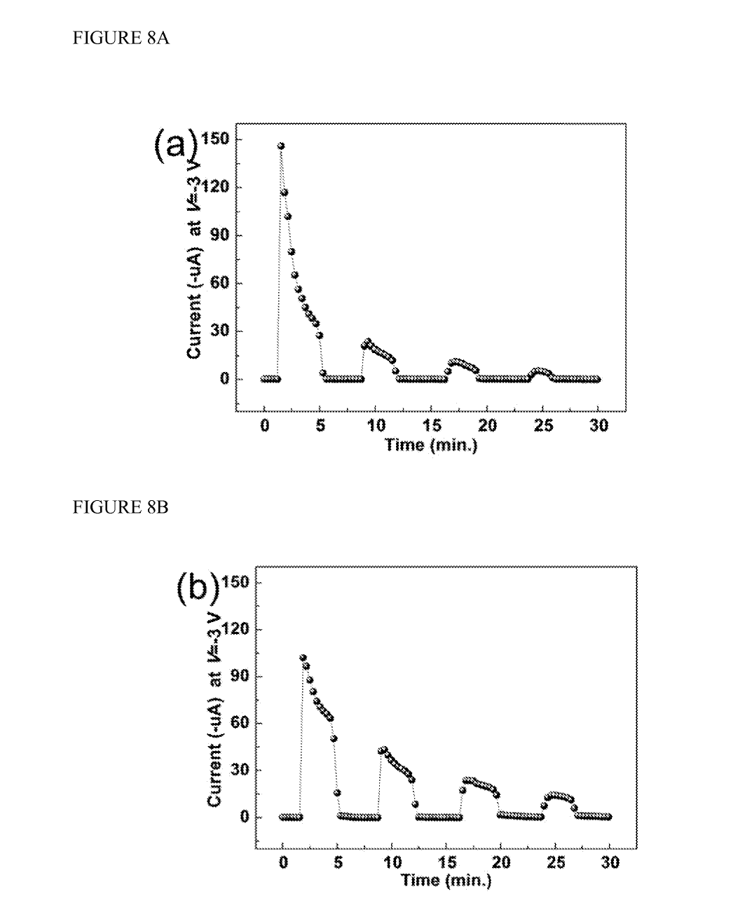

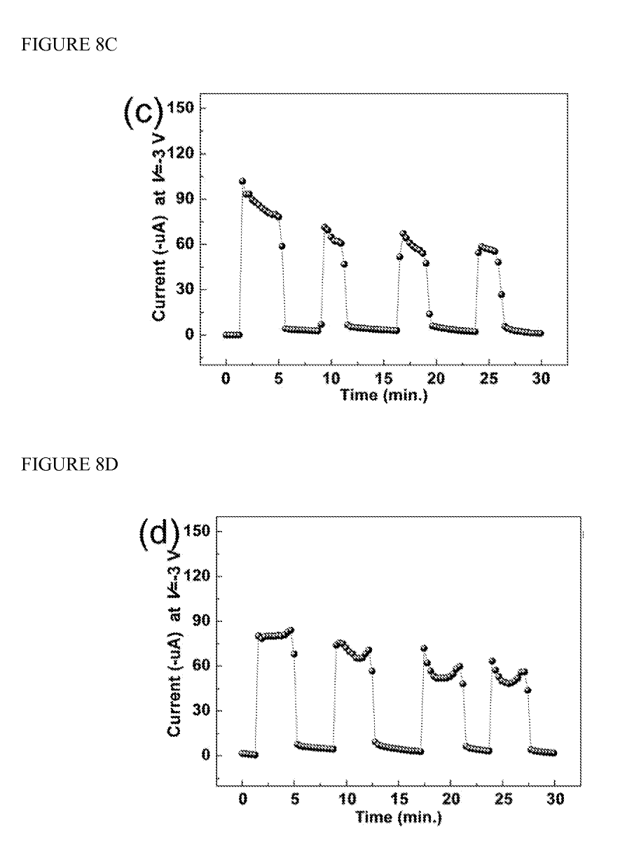

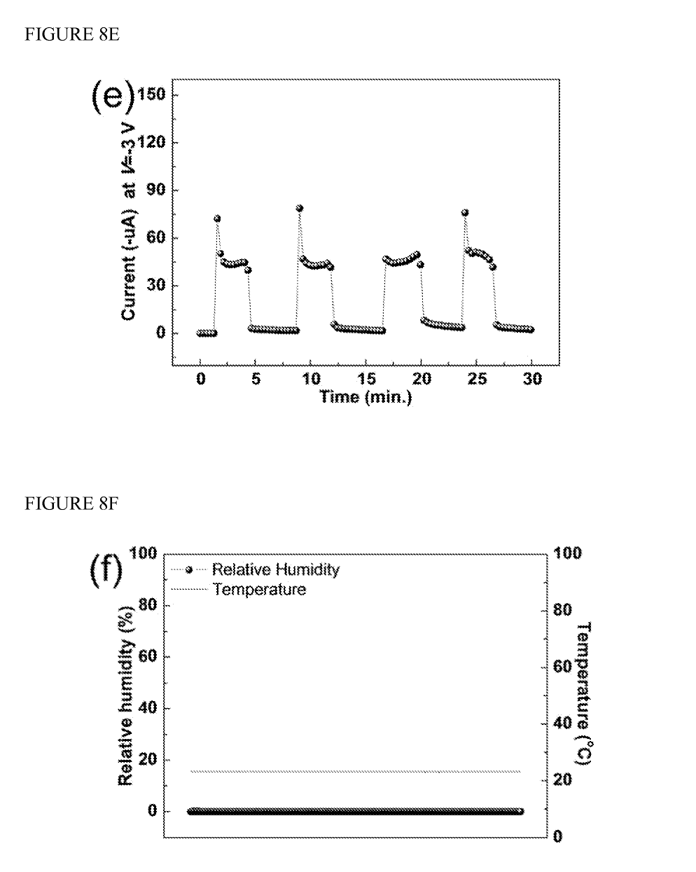

TNT solution of various concentrations was repeatedly dropped four times on the same probed area of each polymer device (FIG. 8). The device exposed to higher concentration TNT solution showed much more increased current from the first drop of TNT solution than from subsequent drops. On the other hand, when the IPA solvent was dropped several times on the same channel area, very similar current changes were reproduced. When the much lower-concentration TNT solution was dropped, the repeated similar current change was observed as for the IPA drop. These results show that the binding of TNT is stable and saturable, even inhibiting the response to the solvent alone. The decreased response for later TNT drops does not reflect a lack of polymer response, but simply the existence of a finite number of active sites that eventually become occupied. This is a possible consequence of the template-induced arrangement of the polymer crosslinks.

### Example 2

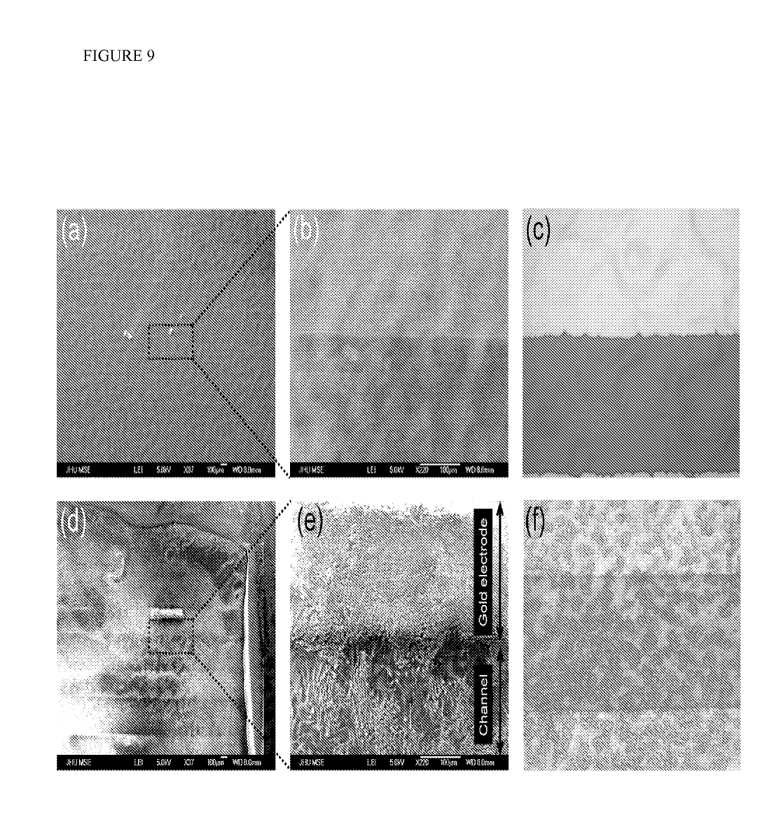

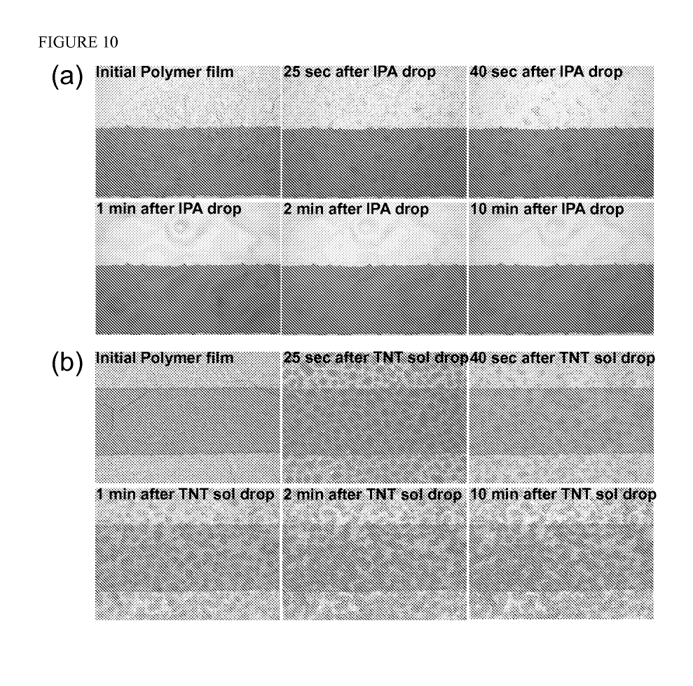

TNT-Induced Morphology Changes. FIG. 9 shows the surface images of films, which had been previously tested electrically, exposed to TNT solution or IPA solvent. The film morphology from a device exposed to the IPA solvent was not changed compared to the initial film. In contrast, when the film was exposed to TNT solution, clearly changed surface images were observed, caused by the reaction mentioned above. In addition, the surface images of the film over time after exposure to the TNT solution or IPA solvent were observed for 10 minutes. (FIG. 10). In the case of the IPA drop, there was no change except for solvent evaporation. However, in the case of the TNT solution drop, the clear formation and movement of domains can be seen which were reacted with TNT analyte.

To obtain atomic force microscopy (AFM) images, 5.0 μL TNT solution or IPA solvent was dropped on the polymer film which had been spincast on a silicon substrate (data not shown). The sample exposed to the IPA solvent showed very similar surface morphology to that of the initial film, including very large crystal domains. However, after exposure to the TNT solution, the film showed a periodic wavy structure with average depth about 4 nm.

The x-ray diffraction (XRD) of the polymer films was additionally measured before and after dropping TNT solution or IPA alone in order to see the changes in the microstructure. The same crystalline peaks were retained after dropping IPA alone, but, after exposure to the TNT analyte, the film became nearly amorphous (FIG. 11).

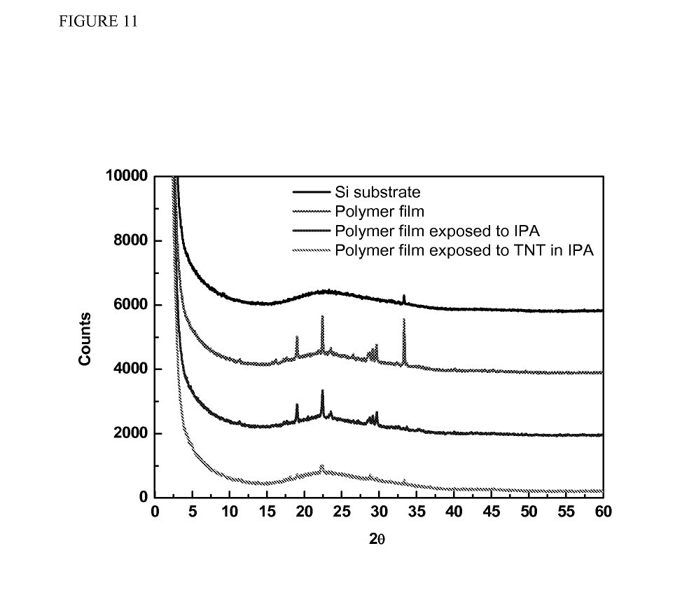

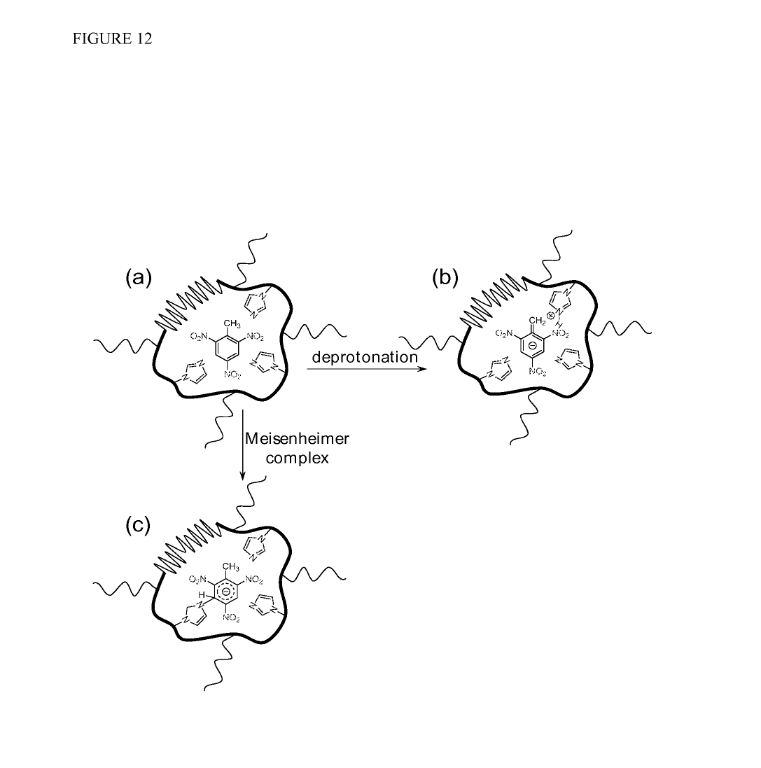

NMR studies and possible reaction mechanism. There are at least two possible types of reactions that could cause these various physical changes. The imidazole groups may interact with TNT through various types of non-covalent bonding mechanisms, including hydrophilic/hydrophobic, electrostatic, hydrogen bonding, and van der Waals forces. Covalent chemistry is also possible (FIG. 12): the imidazole functional groups have pKa values in the range of 6.0-9.0, basic enough to deprotonate the methyl group of TNT or form a Meisenheimer complex with nitroaromatics.

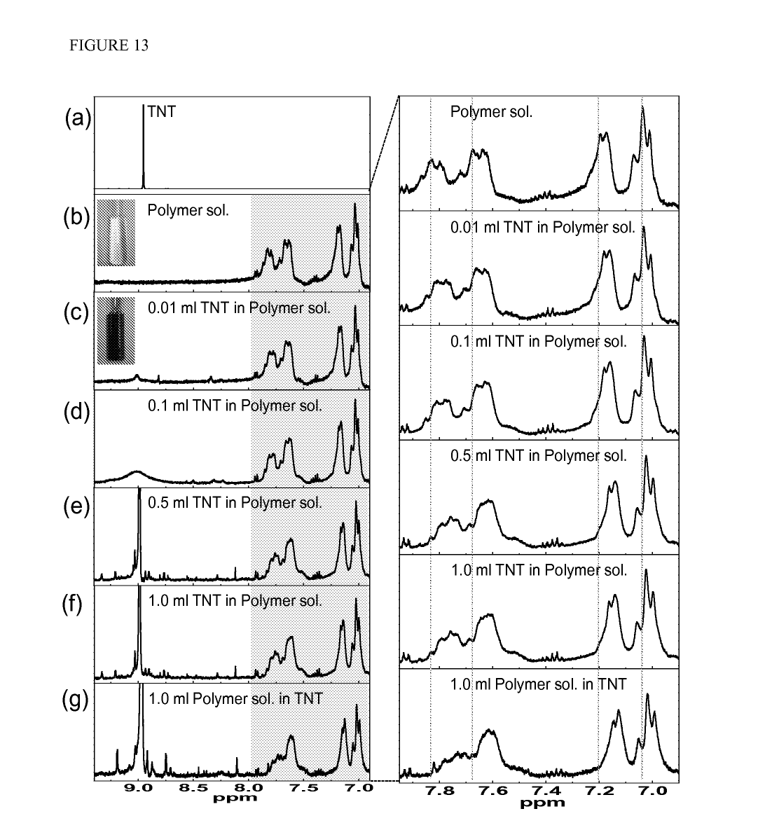

Evidence of these reactions was obtained from ¹H NMR of polymer solutions before and after adding some amount of the TNT analyte (FIG. 13). The solid polymer resin and TNT was dissolved in CD₃OD. Before TNT addition, the imidazole peaks clearly split and the solution color was transparent. As soon as a very small amount of TNT solution (0.01 mL, at the threshold for observation of a distinct TNT peak) was added, the color was very rapidly changed to dark red, the 7.8 ppm imidazole peak was slightly broadened, and all peaks were shifted upfield. When 0.1 mL of TNT solution was added, a broad TNT peak was observed. As more TNT solution was added to the polymer solution, the imidazole peaks were much more broadened and diminished, and shifted further upfield. When a small amount of polymer solution was added to a TNT solution, very similar peak changes were observed. These results verify the strong chemical interactions hypothesized between imidazole groups and TNT in the material.

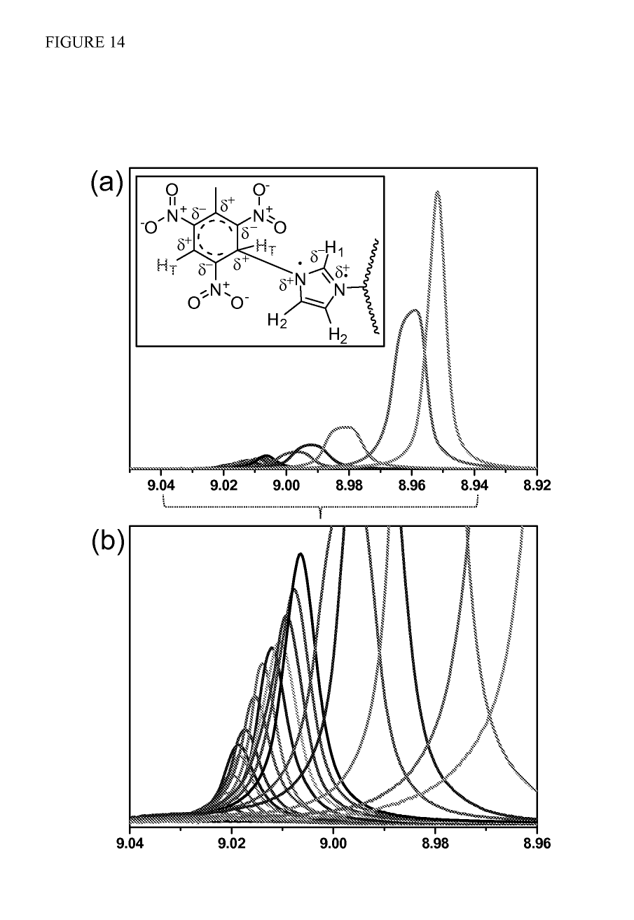

These interactions were quantified by ¹H NMR titration as follows. Increasing amounts of predissolved TNT were added to achieve a range of concentrations of 0 to 0.092 M in a second solution that originally contained 300 mg of polymer in 0.6 mL of CD₃OD. The concentration of imidazole groups in the solution was calculated by relative integration of the imidazole protons compared to the TNT protons, whose concentration was known. The total concentration of free plus complexed imidazole groups decreased as more and more TNT solution was added. The complexation-induced shifts (A6) of the TNT aryl proton (FIG. 14) were monitored and the binding constant for the interaction between the host and analyte was calculated (J. Incl. Phenom. Macro., 2001, 39: 193), assuming that there is a 1:1 binding isotherm, using equation 1.

K=[C]/([T])([M]) Equation 1

where [T]=[T]ₜ−[C] and [C]=[T]ₜ[(δ−δₜ)/(δ_{c}−δₜ)].

[T]ₜ is the total concentration of added TNT, [C] is the concentration of the complex after equilibration, [T] is the concentration of the free TNT after equilibration, [M] is the concentration of free imidazole moieties in the polymer at equilibrium, δ is the observed TNT ¹H chemical shift, and δₜ and δ, are the chemical shifts of TNT in free and totally complexed forms, respectively. δ, was observed either as the onset of the left side of the aryl proton (H_{T}) peak on TNT, or by extrapolation to a single value at zero TNT concentration. With the increase in the concentration of TNT, δ moves upfield; this shielding of H_{T} leads us to conclude that the imidazole forms an adduct with TNT molecule, which is plausibly a Meisenheimer complex, as shown in FIG. 14 (inset). Further addition of TNT makes δ move more towards pure TNT, which is presumably due to the increased weighting of the concentration of free TNT.

The binding constant K for the interaction between the imidazole on the polymer and the TNT was found to be about 53 M⁻¹ with a standard deviation of 8 M⁻¹ arising from nonsystematic uncertainty, when the “onset” method was used to calculate δ_{c}. When various extrapolations to a single value of δ_{c} were used, K between 20 and 60 was obtained, but the error was systematic—higher values were obtained at the lowest TNT concentrations. The listing of data on which these calculations are based is given in the supplementary information. If an extrapolation method is deemed more appropriate, then the data would indicate a suppression of TNT activity at higher concentrations, perhaps from self- or nonspecific aggregation. The “onset” method seems to internally correct for these effects. The binding constant, while modest for dilute solutions, is ample for TNT-imidazole binding and response when little or no solvent is present in an imidazole-rich (0.1 M), low-volume (0.1 μL=1 μm×1 cm²) polymer film, in which case the TNT and imidazole groups are each approximately 50% complexed under the conditions of the experiment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An article of manufacture comprising:
a molecularly-imprinted polymeric material comprising a cross-linked, water-soluble polymer;
at least one imidazole compound, and a binding site capable of selectively binding one or more nitroaromatic compounds;
wherein when the material is contacted with one or more nitroaromatic compounds, increases the conductance of the material when in the presence of an electric current.

2. The article of manufacture of claim 1, wherein the water soluble polymer is comprised of an individual monomer having a single polymerizable group.

3. The article of manufacture of claim 2, wherein the monomer is selected from the group consisting of methylmethacrylate, other alkyl methacrylates, alkylacrylates, ally or aryl acrylates and methacrylates, cyanoacrylate, styrene, α-methyl styrene, vinyl esters, including vinyl acetate, vinyl chloride, methyl vinyl ketone, vinylidene chloride, acrylamide, methacrylamide, acrylonitrile, and methacrylonitrile.

4. The article of manufacture of claim 3, wherein the imidazole compound is N-vinylimidazole.

5. The article of manufacture of claim 3, wherein the article has the polymeric material spin-coated, drop coated, or dip coated onto it.

6. The article of manufacture of claim 5, wherein the article is an electrode.

7. The article of manufacture of claim 5, wherein the article is a chemoresistor.

8. A method of making the article of manufacture of claim 1, comprising:
a) adding to a solvent a sufficient amount of an imidazole compound, the templating nitroaromatic compound, a cosolvent, a cross-linker, and a reversible addition fragmentation chain transfer (RAFT) reagent to make a mixture;
b) adding to the mixture of a), a catalyst at a sufficient concentration;
c) polymerizing the mixture of b) by heating the mixture of b) to a temperature of between 70-80° C. for about 2 to 6 hours; and
d) coating the article with mixture of c).

9. The method of claim 8, wherein the polymer mixture is applied to the article using the methods of spin casting and/or drop casting and/or dip-coating.

10. The method of claim 9, wherein the method for making the polymer mixture further comprises:
d) purifying the polymer of c) by dialysis against water with a molecular weight cutoff of about 3,000-4,000.

11. The method of claim 10, wherein the template is removed from the polymer solution comprising:
e) dialyzing the solution of d) against a sodium bicarbonate buffer; and
f) dialyzing the solution of e) against an aqueous ammonia solution.

12. The article of manufacture of claim 7, wherein the article is a chemoresistor capable of selectively binding one or more nitroaromatic compounds comprising:
a) a silicon substrate; and
b) one or more gold electrodes.

13. A method for detecting the presence of a nitroaromatic compound of interest comprising:
a) providing a sensor comprising an article of manufacture of claim 3: a support substrate; a pair of electrodes comprising (i) a first electrode and (ii) a second electrode, wherein (i) and (ii) comprise the same or different materials, and wherein at least one of (i) and (ii) is at the substrate; wherein the polymeric material is disposed between the first electrode and the second electrode and is bonded to the first electrode and to the second electrode, thereby forming a nanojunction between the first and second electrodes; a detection means operably connected to the pair of electrodes, the detection means capable of detecting a change in the electrical resistance or in the capacitance of the sensor when the polymeric material binds with the nitroaromatic compound of interest;
b) exposing the polymeric material of the sensor to an environment containing nitroaromatic compound of interest such that the nitroaromatic compound of interest contacts the polymeric material;
c) allowing the polymeric material to bind with the nitroaromatic compound of interest, the binding of the nitroaromatic compound of interest resulting in the sensor undergoing a detectable change in electrical resistance or in capacitance; and
d) detecting any change in the electrical resistance or in the capacitance of the sensor, the detected change being indicative of the presence of the nitroaromatic compound of interest in the environment.

14. The method of claim 13, wherein the detection means comprises one or more additional devices comprising the same or a related polymer material not exposed to the nitroaromatic compound.

15. A computerized system identifying one or more nitroaromatic compounds of interest associated with one or more conductance profiles comprising:
a) a server and a client connected by a network;
b) an application connected to the server and/or the client by the network, the application configured for:
c) receiving, by a computer, the conductance profile one or more nitroaromatic compounds of interest from the article of manufacture or chemoresistor of claim 3;
d) comparing by a computer, a library of known conductance profiles of one or more nitroaromatic compounds of interest to the conductance profile from the article of manufacture or chemoresistor described above;
e) identifying, by the computer, the conductance profile from the article of manufacture or chemoresistor described above when it matches a known conductance profiles of one or more nitroaromatic compounds of interest in the library; and
f) transmitting, by the computer, the identified nitroaromatic compound of interest in the chemoresistor to the client.

16. A method for detecting the presence of a nitroaromatic compound of interest comprising:
a) providing a sensor comprising an article of manufacture of claim 3: a support substrate; a pair of electrodes comprising
(i) a first electrode and
(ii) a second electrode, wherein (i) and (ii) comprise the same or different materials, and wherein at least one of (i) and (ii) is at the substrate;
wherein the polymeric material is disposed between the first electrode and the second electrode and is bonded to the first electrode and to the second electrode, thereby forming a nanojunction between the first and second electrodes; a detection means operably connected to the pair of electrodes, the detection means capable of detecting a change in the electrical resistance or in the capacitance of the sensor when the polymeric material binds with the nitroaromatic compound of interest;
b) exposing the polymeric material of the sensor to an environment containing nitroaromatic compound of interest such that the nitroaromatic compound of interest contacts the polymeric material;
c) allowing the polymeric material to bind with the nitroaromatic compound of interest, the binding of the nitroaromatic compound of interest resulting in the sensor undergoing a detectable change in electrical resistance or in capacitance; and
d) detecting any change in the electrical resistance or in the capacitance of the sensor, the detected change being indicative of the presence of the nitroaromatic compound of interest in the environment.
